SEQUENCE LISTING (32 sequences)

SEQ ID NO: 1 - DNA, 1958 bp, Homo sapiens
tcgatctgatttagagatttagggatggatgttttaaaaaaagcaaaagtagtaacagactatagcattggtaatgtgtgtgtgcatatatacatattatttttaaaaaaataaagttcgattatttcacctggcttgtcagtcacctatgcaggcgtctgagcccccgggtttccaggagccccccgtataaggaccccagggactcctctccccacgcggccgggccgcccgcccggcccccagcccggagagctgccaccgaccccctcaacgtcccaagccccagctctgtcgcccgcgttccttcctcttcctgggccacaatcttggctttcccgggccggcttcacgcagttgcgcaggagcccgcgggggaagacctctcgtggggacctcgagcacgacgtgcgaccctaaatccccacatctcctctgccgcctcgcaggccacatgcaccgggagccgggcggggcaggcgcggcccgcaaggacccccgcgatggagacgcaacactgccgcgactgcacttggggcagccccgccgcgtcccagccgcctcccggcaggaagcgtaggtgtgtgagccgacccggagcgagccgcgccctcgggccagcgtgggcagggcgccgcagcctgcgcagccccgaggaccccgcgtcgctctcccgagccagggttctcaggagcgggccgcgcaggagacgttagagggggttgttagcggctgttgggagaacgggtcacggaaacagtcccttccaaagccggggccatcgtggggtgggcgagtccgccctcccaggccgggggcgcggaccagaggggacgtgtgcagacggccgcggtcagccccacctcgcccgggcggagacgcacagctggagctggagggccgtcgcccgttgggccctcaggggcctgaacgcccaggggtcgcggcgagtccacccggagcgagtcaggtgagcaggtcgccatggcgatgcggccccggagagcgcacgcctgccgcggtcggcatggaaacgctcccgctaggtccgggggcgccgctgattggccgattcaacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggatggagaccggctttcccgcaatcatgtaccctggatcttttattgggggctggggagaagagtatctcagctgggaaggaccggggctcccagatttcgtcttccaggtaacgtgggtttagtatcccgacttggaggcttgtcagaatgtttctctccttccagcccaacacgaagtcttgggataaaaagcctccctcagggattcaaataactgttttgattcagagcaactttgatcgcctgtgcggtcgcacctgccctttcagccccaataattactgggaagatcagcaattggtgttagtcccattgcttggtgctctccctcctagaggttcgctgtgtccttggagcccggggtggacggaatcgactaaacagcttgtctgtttctctttccctggtagcagcagcccgtggagtctgaagcaatgcactgcagcaaccccaagagtggagttgtgctggctacagtggcccgaggtcccgatgcttgtcagatactcaccagagccccgctgggccaggat

SEQ ID NO: 2 - DNA, 1442 bp, Homo sapiens
  promoter (1)...(1442)
  mutation (1427)...(1427)
tcgatctgatttagagatttagggatggatgttttaaaaaaagcaaaagtagtaacagactatagcattggtaatgtgtgtgtgcatatatacatattatttttaaaaaaataaagttcgattatttcacctggcttgtcagtcacctatgcaggcgtctgagcccccgggtttccaggagccccccgtataaggaccccagggactcctctccccacgcggccgggccgcccgcccggcccccagcccggagagctgccaccgaccccctcaacgtcccaagccccagctctgtcgcccgcgttccttcctcttcctgggccacaatcttggctttcccgggccggcttcacgcagttgcgcaggagcccgcgggggaagacctctcgtggggacctcgagcacgacgtgcgaccctaaatccccacatctcctctgccgcctcgcaggccacatgcaccgggagccgggcggggcaggcgcggcccgcaaggacccccgcgatggagacgcaacactgccgcgactgcacttggggcagccccgccgcgtcccagccgcctcccggcaggaagcgtaggtgtgtgagccgacccggagcgagccgcgccctcgggccagcgtgggcagggcgccgcagcctgcgcagccccgaggaccccgcgtcgctctcccgagccagggttctcaggagcgggccgcgcaggagacgttagagggggttgttagcggctgttgggagaacgggtcacggaaacagtcccttccaaagccggggccatcgtggggtgggcgagtccgccctcccaggccgggggcgcggaccagaggggacgtgtgcagacggccgcggtcagccccacctcgcccgggcggagacgcacagctggagctggagggccgtcgcccgttgggccctcaggggcctgaacgcccaggggtcgcggcgagtccacccggagcgagtcaggtgagcaggtcgccatggcgatgcggccccggagagcgcacgcctgccgcggtcggcatggaaacgctcccgctaggtccgggggcgccgctgattggccgattcaacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 3 - DNA, 1294 bp, Homo sapiens
  promoter (1)...(1294)
  mutation (1279)...(1279)
atgcaggcgtctgagcccccgggtttccaggagccccccgtataaggaccccagggactcctctccccacgcggccgggccgcccgcccggcccccagcccggagagctgccaccgaccccctcaacgtcccaagccccagctctgtcgcccgcgttccttcctcttcctgggccacaatcttggctttcccgggccggcttcacgcagttgcgcaggagcccgcgggggaagacctctcgtggggacctcgagcacgacgtgcgaccctaaatccccacatctcctctgccgcctcgcaggccacatgcaccgggagccgggcggggcaggcgcggcccgcaaggacccccgcgatggagacgcaacactgccgcgactgcacttggggcagccccgccgcgtcccagccgcctcccggcaggaagcgtaggtgtgtgagccgacccggagcgagccgcgccctcgggccagcgtgggcagggcgccgcagcctgcgcagccccgaggaccccgcgtcgctctcccgagccagggttctcaggagcgggccgcgcaggagacgttagagggggttgttagcggctgttgggagaacgggtcacggaaacagtcccttccaaagccggggccatcgtggggtgggcgagtccgccctcccaggccgggggcgcggaccagaggggacgtgtgcagacggccgcggtcagccccacctcgcccgggcggagacgcacagctggagctggagggccgtcgcccgttgggccctcaggggcctgaacgcccaggggtcgcggcgagtccacccggagcgagtcaggtgagcaggtcgccatggcgatgcggccccggagagcgcacgcctgccgcggtcggcatggaaacgctcccgctaggtccgggggcgccgctgattggccgattcaacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 4 - DNA, 597 bp, Homo sapiens
  promoter (1)...(597)
  mutation (582)...(582)
ggccgcggtcagccccacctcgcccgggcggagacgcacagctggagctggagggccgtcgcccgttgggccctcaggggcctgaacgcccaggggtcgcggcgagtccacccggagcgagtcaggtgagcaggtcgccatggcgatgcggccccggagagcgcacgcctgccgcggtcggcatggaaacgctcccgctaggtccgggggcgccgctgattggccgattcaacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 5 - DNA, 469 bp, Homo sapiens
  promoter (1)...(469)
  mutation (454)...(454)
agcaggtcgccatggcgatgcggccccggagagcgcacgcctgccgcggtcggcatggaaacgctcccgctaggtccgggggcgccgctgattggccgattcaacagacgcgggtgggcagctcagccgcatcgctaagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 6 - DNA, 333 bp, Homo sapiens
  promoter (1)...(333)
  mutation (318)...(318)
aagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 7 - DNA, 303 bp, Homo sapiens
  promoter (1)...(303)
  mutation (288)...(288)
tcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 8 - DNA, 263 bp, Homo sapiens
  promoter (1)...(263)
  mutation (248)...(248)
cccttccctgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 9 - DNA, 255 bp, Homo sapiens
  promoter (1)...(255)
  mutation (240)...(240)
tgcccttccccgccctgccccgcccggcccggcccggccctgcccaaccctgccccgccctgccccgcccagccggccacctcttaaccgcgatcctccagtgcacttgccagttgttccggacacatagaaagataacgacgggaagacggggccccgtttggggtccaggcaggttttggggcctcctgtctggtgggaggaggccgcagcgcagcaccctgctcgtcacttgggattgagaccggctttccc

SEQ ID NO: 10 - DNA, 209 bp, Homo sapiens
  promoter (1)...(209)
aagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggacacataga

SEQ ID NO: 11 - DNA, 202 bp, Homo sapiens
  promoter (1)...(202)
aagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgttccggac

SEQ ID NO: 12 - DNA, 195 bp, Homo sapiens
  promoter (1)...(195)
aagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttgt

SEQ ID NO: 13 - DNA, 194 bp, Homo sapiens
  promoter (1)...(194)
aagcccggccgcctcccaggctggaatccctcgacacttggtccttcccgccccgcccttccgtgccctgcccttccctgcccttccccgccctgccccgcccggcccggcccggccctgcccaaccctgccccgccctgccccgcccagccggccacctcttaaccgcgatcctccagtgcacttgccagttg

SEQ ID NO: 14 - DNA, 21 bp, Artificial Sequence
  Single-stranded oligonucleotide primer sequence
ccctctcagaacagacataca

SEQ ID NO: 15 - DNA, 21 bp, Artificial Sequence
  Single-stranded oligonucleotide primer sequence
ctgatccagaataacacctga

SEQ ID NO: 16 - DNA, 36 bp, Artificial Sequence
  n equals inosine; Universal 5′ RACE abridged anchor primer
ggccacgcgtcgactagtacgggnngggnngggnng

SEQ ID NO: 17 - DNA, 19 bp, Artificial Sequence
  Single-stranded oligonucleotide primer sequence
ctcctcccaccagacagga

SEQ ID NO: 18 - DNA, 26 bp, Artificial Sequence
  Double-stranded oligonucleotide
cctgccccgccctgccccgcccagcc

SEQ ID NO: 19 - DNA, 27 bp, Artificial Sequence
  Double-stranded oligonucleotide
ccttccccgccctgccccgcccggccc

SEQ ID NO: 20 - DNA, 22 bp, Artificial Sequence
  Double-stranded oligonucleotide
attcgatcggggcggggcgagc

SEQ ID NO: 21 - DNA, 25 bp, Artificial Sequence
  Double-stranded oligonucleotide
gagaccggctcgaacgcaatcatgt

SEQ ID NO: 22 - DNA, 24 bp, Artificial Sequence
  Single-stranded oligonucleotide
aatcgataagaattcgtcgaccga

SEQ ID NO: 23 - DNA, 29 bp, Artificial Sequence
  Single-stranded oligonucleotide
ccccgccctgccccttacagccggccacc

SEQ ID NO: 24 - DNA, 29 bp, Artificial Sequence
  Single-stranded oligonucleotide
ccccgccctgccccttacagccggccacc

SEQ ID NO: 25 - DNA, 22 bp, Artificial Sequence
  Single-stranded oligonucleotide
ccctgccccttccggcccggcc

SEQ ID NO: 26 - DNA, 22 bp, Artificial Sequence
  Single-stranded oligonucleotide
ctgcccttcccttccctgcccc

SEQ ID NO: 27 - DNA, 42 bp, Artificial Sequence
  Single-stranded oligonucleotide
gcccaaccctgcccttacctgccccttacagccggccacctc

SEQ ID NO: 28 - DNA, 45 bp, Artificial Sequence
  Single-stranded oligonucleotide
cttccctgcccttcccttacctgccccttacggcccggcccggcc

SEQ ID NO: 29 - DNA, 29 bp, Artificial Sequence
  Single-stranded oligonucleotide
ccacctcttaacaagcttcctccagtgca

SEQ ID NO: 30 - DNA, 24 bp, Artificial Sequence
  EGFP-specific single-stranded oligonucleotide
ttgtcgggcagcagcacggggccg

SEQ ID NO: 31 - DNA, 24 bp, Artificial Sequence
  EGFP-specific single-stranded oligonucleotide
tcaccggggtggtgccatcctttg

SEQ ID NO: 32 - DNA, 6 bp, Homo sapiens
  misc_feature (0)...(0)
tcgcgg

(19) United States
(12) Patent Application Publication
(10) Pub. No.: US 2002/0056148 A1
(43) Pub. Date: May 9, 2002

(54) Transfection, storage and transfer of male germ cells for generation of selectable transgenic stem cells

(76) Inventors: Carol W. Readhead, Pasadena, CA (US); Robert Winston, London (GB); H. Phillip Koeffler, Los Angeles, CA (US); Carsten Muller, Los Angeles, CA (US)

Correspondence Address: Edward G. Poplawski, Esq., Sidley & Austin, A Partnership including Professional Corporation, 555 West Fifth Street, Los Angeles, CA, 90013-1010, US

(21) Appl. No.: 09/919,042

(22) Filed: Jul. 30, 2001

Related U.S. Application Data
(62) Division of application No. 09/292,723, filed on Apr. 15, 1999.
Application No. 09/292,723 is a continuation-in-part of application No. 09/191,920, filed on Nov. 13, 1998, now Pat. No. 6,316,692.
(60) Provisional application No. 60/065,825, filed on Nov. 14, 1997.

(30) Foreign Application Priority Data
Nov. 13, 1998 (US) PCT/US98/24238

(51) Int. Cl. A01K 67/027; C12N 5/06; C12N 5/08

(52) U.S. Cl. 800/14; 800/19; 435/325; 435/366; 435/349

(57) ABSTRACT

Disclosed is a method of obtaining selectable transgenic stem cells of a vertebrate by transfecting a male germ cell with a transfection mixture comprising a nucleic acid construct containing a transcriptional unit of a stem cell-specific promoter, for example, a cyclin A1 promoter, operatively linked to a gene encoding a fluorescent or light-emitting reporter protein. The transfection mixture is a composition for transfection, in vivo or ex vivo, of a vertebrate's male germ cells, which comprises a nucleic acid or transgene, and a gene delivery system, and optionally a protective internalizing agent, such as an endosomal lytic agent, a virus or a viral component, which is internalized by cells along with the transgene and which enhances gene transfer through the cytoplasm to the nucleus of the male germ cell. In stem cells, other than germ cells, grown in vivo, expression of the reporter gene from a cyclin A1 promoter is facilitated by preventing methylation of promoter DNA by the use of flanking insulator elements in the nucleic acid construct. Alternatively, inhibitors of DNA methylation can be used in an in vitro growth medium. A kit contains components of the transfection mixture. Selectable transgenic stem cells have stably integrated the DNA, and non-human transgenic vertebrates comprise these selectable transgenic stem cells.

This application is a continuation-in-part of U.S. patent application Ser. No. 09/191,920, filed Nov. 13,1998, which claims the benefit of U.S. Provisional Application No. 60/065825, filed on Nov. 14, 1997.

### BACKGROUND OF THE INVENTION

Throughout this application various publications are referenced within parentheses. The disclosures of these publications in their entireties are hereby incorporated by reference in this application in order to more fully describe the state of the art to which this invention pertains.

1. The Field of the Invention

This invention relates to the medical arts, particularly to the field of transgenics and gene therapy. The invention is particularly directed to the field of transgenic vertebrate stem cells.

2. Discussion of the Related Art

The field of transgenics was initially developed to understand the action of a single gene in the context of the whole animal and phenomena of gene activation, expression, and interaction. This technology has been used to produce models for various diseases in humans and other animals. Transgenic technology is among the most powerful tools available for the study of genetics, and the understanding of genetic mechanisms and function. It is also used to study the relationship between genes and diseases. About 5,000 diseases are caused by a single genetic defect. More commonly, other diseases are the result of complex interactions between one or more genes and environmental agents, such as viruses or carcinogens. The understanding of such interactions is of prime importance for the development of therapies, such as gene therapy and drug therapies, and also treatments such as organ transplantation. Such treatments compensate for functional deficiencies and/or may eliminate undesirable functions expressed in an organism. Transgenesis has also been used for the improvement of livestock, and for the large scale production of biologically active pharmaceuticals.

Historically, transgenic animals have been produced almost exclusively by micro injection of the fertilized egg. The pronuclei of fertilized eggs are micro-injected in vitro with foreign, i.e., xenogeneic or allogeneic DNA or hybrid DNA molecules. The micro-injected fertilized eggs are then transferred to the genital tract of a pseudopregnant female. (E.g., P. J. A. Krimpenfort et al., Transgenic mice depleted in mature T-cells and methodsfor making transgenic mice, U.S. Pat. Nos. 5,175,384 and 5,434,340; P. J. A. Krimpenfort et al., Transgenic mice depleted in mature lymphocytic cell-type, U.S. Pat. No. 5,591,669).

The generation of transgenic animals by this technique is generally reproducible, and for this reason little has been done to improve on it. This technique, however, requires large numbers of fertilized eggs. This is partly because there is a high rate of egg loss due to lysis during micro-injection. Moreover manipulated embryos are less likely to implant and survive in utero. These factors contribute to the technique's extremely low efficiency. For example, 300-500 fertilized eggs may need to be micro injected to produce perhaps three transgenic animals. Partly because of the need to micro-inject large numbers of embryos, transgenic technology has largely been exploited in mice because of their high fecundity. While small animals such as mice have proved to be suitable models for certain diseases, their value in this respect is limited. Larger animals would be much more suitable to study the effects and treatment of most human diseases because of their greater similarity to humans in many aspects, and also the size of their organs. Now that transgenic animals with the potential for human xenotransplantation are being developed, larger animals, of a size comparable to man will be required. Transgenic technology will allow that such donor animals will be immunocompatible with the human recipient. Historical transgenic techniques, however, require that there be an ample supply of fertilized female germ cells or eggs. Most large mammals, such as primates, cows, horses and pigs produce only 10-20 or less eggs per animal per cycle even after hormonal stimulation. Consequently, generating large animals with these techniques is prohibitively expensive.

This invention relies on the fact that spermatogenesis in male vertebrates produces vast numbers of male germ cells that are more readily available than female germ cells. Most male mammals generally produce at least 10⁸ spermatozoa (male germ cells) in each ejaculate. This is in contrast to only 10-20 eggs in a mouse even after treatment with superovulatory drugs. A similar situation is true for ovulation in nearly all larger animals. For this reason alone, male germ cells will be a better target for introducing foreign DNA into the germ line, leading to the generation of transgenic animals with increased efficiency and after simple, natural mating.

Spermatogenesis is the process by which a diploid spermatogonial stem cell provides daughter cells which undergo dramatic and distinct morphological changes to become self-propelling haploid cells (male gametes) capable, when fully mature, of fertilizing an ovum.

Primordial germ cells are first seen in the endodermal yolk sac epithelium at E8 and are thought to arise from the embryonic ectoderm (A. McLaren and Buehr, Cell Diff. Dev. 31:185 [1992]; Y. Matsui et al., Nature 353:750 [1991]). They migrate from the yolk sac epithelium through the hindgut endoderm to the genital ridges and proliferate through mitotic division to populate the testis.

At sexual maturity the spermatogonium goes through 5 or 6 mitotic divisions before it enters meiosis. The primitive spermatogonial stem cells (Ao/As) proliferate and form a population of intermediate spermatogonia types Apr, Aal, Al-4 after which they differentiate into type B spermatogonia. The type B spermatogonia differentiate to form primary spermatocytes which enter a prolonged meiotic prophase during which homologous chromosomes pair and recombine. The states of meiosis that are morphologically distinguishable are; preleptotene, leptotene, zygotene, pachytene, secondary spermatocytes and the haploid spermatids. Spermatids undergo great morphological changes during spermatogenesis, such as reshaping the nucleus, formation of the acrosome and assembly of the tail (A. R. Bellve et al., Recovery, capacitation, acrosome reaction, and fractionation of sperm, Methods Enzymol. 225:113-36 [1993]). The spermatocytes and spermatids establish vital contacts with the Sertoli cells through unique hemi-junctional attachments with the Sertoli cell membrane. The final changes in the maturing spermatozoan take place in the genital tract of the female prior to fertilization.

Initially, attempts were made to produce transgenic animals by adding DNA to spermatozoa which were then used to fertilize mouse eggs in vitro. The fertilized eggs were then transferred to pseudopregnant foster females, and of the pups born, 30% were reported to be transgenic and express the transgene. Despite repeated efforts by others, however, this experiment could not be reproduced and no transgenic pups were obtained. Indeed, there remains little doubt that the transgenic animals reputed to have been obtained by this method were not transgenic at all and the DNA incorporation reported was mere experimental artifact. Data collected from laboratories around the world engaged in testing this method showed that no transgenics were obtained from a total of 890 pups generated.

In summary, it is currently possible to produce live transgenic progeny but the previously available methods are costly and extremely inefficient. Therefore, there is a definite need for a simple, less costly and less invasive method of producing transgenic animals.

There has also been a need for a way of selecting or isolating stem cells from non-stem cells, for study or therapeutic uses, that does not require the use of embryonic material, because the use of embryonic material may present ethical problems. In addition, the study of stem cells specifically in the physiologic milieu of non-embryonic (e.g., adult) vertebrates has been hampered by the difficulty of selecting, identifying, or isolating stem cells from non-stem cells in the tissues of these organisms.

A stem cell is an undifferentiated mother cell that is self-renewable over the life of the organism and is multipotent, i.e., capable of generating various committed progenitor cells that can develop into fully mature differentiated cell lines. (T. Zigova and P. R. Sanberg, The rising star of neural stem cell research, Nature Biotechnol. 16(11):1007-08 [1998]). All vertebrate tissues arise from stem cells, including hematopoietic stem cells, from which various types of blood cells derive; neural stem cells, from which brain and nerve tissues derive; and germ cells, from which male or female gametes derive.

Recently, there has been a great deal of interest in transgenic stem cells as a potential therapeutic tool for patients suffering from genetic diseases, metabolic defects, varying kinds oftrauma, diseases of the nervous system, or cancers of the blood. In manipulating stem cells in vitro or in vivo it is important to be able to identify and select stem cells of interest from non-stem cells.

Tsukamoto et al. disclosed a method for identifying human hematopoietic stem cells based on specific antibody binding to Thy-1 and CD34 surface epitopes. (A. Tsukamoto et al., Identification and isolation of human hematopoietic stem cells, U.S. Pat. No. 5,643,741). Tsukamoto et al. taught embodiments of their method in which the antibodies are labeled with a fluorochrome and detection of stem cells is by fluorescence activated cell sorter (FACS). Murray et al. taught a method of purifying a population of hematopoietic stem cells expressing a CDw109 marker that used binding of monoclonal antibodies specific for Cdw109. (L. Murray et al., Method of purifying a population of cells enriched for hematopoietic stem cells, populations of cells obtained thereby and methods of use thereof, U.S. Pat. No. 5,665,557).

Transgenic neural stem cells (NSCs) have also been identified and selected using immunofluorescence or other immunostaining techniques. (J. D. Flax et al., Engraftable human neural stem cells respond to developmental cues, replace neurons, and express foreign genes, Nature Biotechnol. 16(11):1033-39 [1998]; O. Bruestle et al., Chimeric brains generated by intraventricular transplantation of fetal human brain cells into embryonic rats, Nature Biotechnol. 16(11):1040-44 [1998]).

However, such immunologically based methods as these have limited usefulness in identifying or selecting stem cells, because they rely on tissue- or lineage-specific epitopes and do not consistently leave the cells in a viable condition. Others have addressed the latter problem using non-lethal methods for labeling transgenic cells, particularly using genes encoding fluorescent or bioluminescent markers. For example, Chalfie et al. disclosed a recombinant DNA molecule comprising the green fluorescent protein gene operatively linked to any exogenous regulatory element. (M. Chalfie et al., Uses of green-fluorescent protein, U.S. Pat. No. 5,491,084). Cormier et al. taught a recombinant DNA vector comprising the gene for apoaequorin, a bioluminescent protein. (M. J. Cormier et al., Recombinant DNA vectors capable of expressing apoaequorin, U.S. Pat. No. 5,422,266).

Contag et al. disclosed a method for detecting a transformed cell of interest expressing a light-generating moiety in vivo, (C. H. Contag, Non-invasive localization of a light-emitting conjugate in a mammal, U.S. Pat. No.5,650,135). Similarly, Horan et aL disclosed a method for tracking cells in vivo related to labeling cells with a fluorecent cyanine dye. (P. K. Horan et al., In vivo cellular tracking, U.S. Pat. No. 4,762,701). And Patterson et al. taught a method of detecting cells expressing a specific nucleotide target sequence by using fluorescently labeled complementary nucleic acid probes and fluorescence-activated flow cytomety (FACS). (Patterson et al., Method of detecting amplified nucleic sequences in cells by flow cytometry, U.S. Pat. No. 5,840,478).

Lineage specific stem cell promoters and other regulatory elements are available that could be linked to the expression of a marker gene. For example, Burn et al. taught the use of a CD34 promoter, specific to hematopoietic stem cells. (T. C. Burn et al., Hematopoietic stem cell specific gene expression, U.S. Pat. No. 5,556,954).

Gay disclosed a method of isolating a lineage specific stem cell in vitro. (D. A. Gay, Method of isolating a lineage specific stem cell in vitro, U.S. Pat. No. 5,639,618). The method involved in vitro transfection of pluripotent embryonic stem cells with a construct comprising a lineage specific promoter sequence operably linked to a DNA encoding a fluorescent or other reporter protein. But this method was not applicable in a generalized way to selecting stem cells in vitro or in vivo in transgenic animals. For this purpose, there has been a definite need for a promoter sequence that operates in a wide variety of stem cells, rather than regulating transcription in a lineage specific manner.

The differentiation of stem cells into somatic cells as well as normal cell growth depend on the regulation of the cell cycle. Dysfunction of this regulation can lead to uncontrolled cell growth and cancer (L. H. Hartwell and M. B. Kastan, Cell cycle control and cancer, Science 266:1821-28 [1994]). Important in the regulation of growth and differentiation are the cyclins. Cyclins are positive regulators of cyclin-dependent kinases (CDKs), with which they can form activated complexes that play a central role in driving the cell through the cell cycle. The activities of these CDK's are regulated by sequential activating and inactivating phosphorylation and de-phosphorylation events. (D. O. Morgan, Principles of CDK regulation, Nature (Lond.) 374:131-34 [1995]; C. J. Sherr, Phase progression: cycling on cue, Cell 79:551-555 [1994]; P. Nurse, Ordering S phase and M phase in the cell cycle, Cell 79:547-50 [1994]). Negative regulators called CDK inhibitors can bind to and inhibit CDK's, adding another layer of regulation (T. Hirama and H. P. Koeffler, Role of the cyclin-dependent kinase inhibitors in the development of cancer, Blood 86:841-54 [1995]; C. J. Sherr and J. M. Roberts, Inhibitors of mammalian G1 cyclin-dependent kinases, Genes Dev. 9:1149-1163 [1995]).

The kinase activity of the cyclin A/CDK2 complex, which rises at the G₁ to S transition, is required for entry into S phase (K. A. Heichman and J. M. Roberts, Rules to replicate by, Cell 79:557-62 [1994]; M. Pagano et al., Cyclin A is required at two points in the human cell cycle, EMBO J. 11:961-71 [1992]; J. Pines and T. Hunter, Human cyclin A is adenovirus E1A-associated protein p60 and behaves differently from cyclin B, Nature 346:760-63 [1990]; C. Desdouets et al., Cyclin A: function and expression during cell proliferation, Prog. Cell Cycle Res. 1:15-23 [1995]). Cyclin A also forms a complex with CDC2, the activity of which peaks at the G₂ to M transition, and the kinase activity of cyclin A/CDC2 is also required for M-phase entry (M. Pagano et al. [1992]).

Two kinds of cyclin A were first found in Xenopus; early embryos contained both cyclin A1 and cyclin A2. Later in development, cyclin A2, which shares considerable homology to mammalian cyclin A2, was found throughout the embryo, whereas cyclin A1 was found only in the testis and ovary. (J. A. Howe et al., Identification of a developmental timer regulating the stability of embryonic cyclin A and a new somatic A-type cyclin at gastrulation, Genes Dev. 9(10): 164-76 [1995]). In the mouse, cyclin A2 was found in a number of tissues during development, but cyclin A1 expression was highly restricted, with high levels measured in late pachytene spermatocytes. (C. Sweeney et al., A distinct cyclin A is expressed in germ cells in the mouse, Development 122(1):53-64 [1996]).

Cyclin A1 is not expressed in fully differentiated cells of non-embryonic tissues, but can be expressed in a wide variety of stem cells, including male and female germ cells, brain stem cells, hematopoietic progenitor cells, as well as in a majority of myeloid leukemic cells and undifferentiated hematological malignancies. (R. Yang et al., Characterization of a second human cyclin A that is highly expressed in testis and in several leukemic cell lines, Cancer Res. 57(5):913-20 [1997]; A. Kramer et al., Cyclin A1 is predominantly expressed in hematological malignancies with myeloid differentiation, Leukemia 12(6):893-98 [1998]; C. Sweeney et al. [1996]; J. A. Howe et al. [1995]). The pattern of cyclin A1 expression indicates that its regulation differs from that of cyclin A2, and this may be related to differential binding by cyclin A1 and cyclin A2 promoters of transcriptional initiation factors, such as the Sp 1 family of initiation factors.

The Sp1 family of initiation factors is related to the regulation of differentiation in stem cells. (K. L. Block et al., Blood 88:2071-80 [1996]; H. M. Chen et al., J. Biol. Chem. 268:8230-39 [1993]; R. K. Margana et al., J. Biol. Chem. 272:3083-90 [1997]). Sp1 is expressed at high levels in tissues where cyclin A1 expression is found. (C. Sweeney et al. [1996]). Also, induction of Sp1 was found to be associated with differentiation of embryonal carcinoma cells and Sp1 was causally linked to expression of the fibronectin gene, providing evidence for a role of Sp1 in differentiation. (M. Suzuki et al., Molecular & Cellular Biology 18: 3010-3020 [1998]). In adult tissue, high levels of Sp1 have been reported in hematopoietic progenitors and in the later stages of spermatogenesis. (J. D. Safer et al., Molecular & Cellular Biology 11: 2189-2199 [1991]).

Levels of Sp1 vary up to 10-fold in different tissues. (J. D.Safer et al. [1991]). This could provide a basis for directing tissue specific expression in stem cells, especially if the affinity of the cis-acting Sp1 family binding sites of various promoters differ. Another mechanism of tissue-directed expression depends on the molar ratios of Sp1 family members to each other resulting in either activation or repression of transcription. (A. P. Kumar et al., Nucleic Acids Res. 25:2012-19 [1997]; M. J. Birnbaum et al., Biochem. 34:16503-08 [1995]).

Sp1 has been shown to serve distinct roles in transcriptional activation: it can directly interact with the basal transcription complex. (A. Emili et al., Molec. Cell. Biol. 14:1582-93 [1994]) and it can determine the transcription start site in TATA-less promoters (J. Lu et al., J. Biol. Chem. 269:5391-5402 [1994]). However, Sp1 can also function as a more general transcriptional activator, and an Sp1 family member, Sp3 protein, is known to function either as transcriptional activator or repressor depending on the context of the binding site in a promoter. (D. Apt et al., Virol. 224:281-91 [1996]; B. Majello et al., J. Biol. Chem. 272:4021-26 [1997]). When Sp3 binds to a single site, it can activate transcription but binding to multiple sites can lead to strong transcriptional repression (M. J. Birnbaum et al., Biochem. 34:16503-08 [1995]).

Also, since myb was shown to be expressed in male germs cells, myb probably acts as an important transcriptional factor for expression from the cyclin A1 promoter during spermatogenesis as well as hematopoiesis. (J. Sitzmann et al., Expression of B-Myb during mouse embryogenesis, Oncogene 12:1889-94 [1996]; K. Latham et al., Oncogene 13:1161-68 [1998]). The structure of myb protein includes a helix-turn-helix motif involved with DNA recognition. (M. D. Carr et aL, Eur. J. Biochem. 235:721-735 [1996]). The myb proteins bind DNA as monomers, with cooperative binding of the R2 and R3regions within the major groove to the consensus myb binding site, MBS (c/TAAcNG). (K. M. Howe and R. J. Watson, ENMBO J. 9:161-69 [1990]; K. Ogata et al., Nature Struct. Biol. 2:309-20 [1995]). The precise role of myb transcription factors in cell cycle regulation is unknown but as a transcriptional activator they may be important for the activation of cell cycle genes such as cyclin A1. (Reviews: S. A. Ness, BiochimBiophys. Acta 1288:F123-F139 [1996]; M. K. Saville and R. J. Watson, Adv. Cancer Res. 72:109-40 [1998]).

The present invention addresses the need for spermatogenic transfection, either in vitro or in vivo, that is highly effective in transferring allogeneic as well as xenogeneic genes into the animal's germ cells and in producing transgenic vertebrate animals. The present technology addresses the requirements of germ line and stem cell line gene therapies in humans and other vertebrate species. Further, the method of the present invention particularly addresses the problem of identifying and selecting stem cells from non-stem cells including differentiated somatic cells, especially from non-embryonic biological sources.

These and other benefits and features of the present invention are described herein.

### SUMMARY OF THE INVENTION

The present invention relates to the in vivo and ex vivo (in vitro) transfection of eukaryotic animal germ cells with a desired genetic material. Briefly, the in vivo method involves injection of genetic material together with a suitable vector directly into the testicle of the animal. In this method, all or some of the male germ cells within the testicle are transfected in situ, under effective conditions. The ex vivo method involves extracting germ cells from the gonad of a suitable donor or from the animal's own gonad, using a novel isolation method, transfecting them in vitro, and then returning them to the testis under suitable conditions where they will spontaneously repopulate it. The ex vivo method has the advantage that the transfected germ cells may be screened by various means before being returned to the testis to ensure that the transgene is incorporated into the genome in a stable state. Moreover, after screening and cell sorting only enriched populations of germ cells may be returned. This approach provides a greater chance of transgenic progeny after mating.

This invention also relates to a novel method for the isolation of spermatogonia, comprising obtaining spermatogonia from a mixed population of testicular cells by extruding the cells from the seminiferous tubules and gentle enzymatic disaggregation. The spermatogonia or stem cells which are to be genetically modified, may be isolated from a mixed cell population by a novel method including the utilization of a promoter sequence, which is only active in stem cells, for example the cyclin A1 promoter, or in cycling spermatogonial stem cell populations, for example, B-Myb promoter or a spermotogonia specific promoter, such as the c-kit promoter region, c-raf-1 promoter, ATM (ataxia-telangiectasia) promoter, RBM (ribosome binding motif) promoter, DAZ (deleted in azoospermia) promoter, XRCC-1 promoter, HSP 90 (heat shock gene) promoter, or FRMI (from fragile X site) promoter, optionally linked to a reporter construct, for example, a construct encoding Green Fluorescent Protein (EGFP), Yellow Fluorescent Protein, Blue Fluorescent Protein, a phycobiliprotein, such as phycoerythrin or phycocyanin, or any other protein which fluoresces under suitable wave-lengths of ultraviolet light. These unique promoter sequences drive the expression of the reporter construct only in the cycling spermatogonia or stem cells in which they operate. The spermatogonia or stem cells, thus, are the only cells in the mixed population which will express the reporter construct and they, thus, may be isolated on this basis. Transgenic cells expressing a fluorescent reporter construct can be sorted with the aid of, for example, a flow activated cell sorter (FACS) set at the appropriate wavelength or they may be selected by chemical methods.

The present invention also relates to a method of obtaining selectable transgenic stem cells by transfecting a male germ cell with a DNA construct comprising a stem cell-specific promoter, for example, a cyclin A1 promoter, operatively linked to a gene encoding a fluorescent or light-emitting reporter protein. The present invention also relates to selectable transgenic stem cells that have stably integrated the DNA and non-human transgenic vertebrates comprising them. In stem cells other than germ cells, expression of the reporter gene from a cyclin A1 promoter in vivo is facilitated by preventing the methylation of promoter DNA by the use of flanking insulator elements. Alternatively, when transgenic stem cells are grown in vitro, inhibitors of DNA methylation can be added to the culture medium.

For transfection, the method of the invention comprises administering to the animal, or to germ cells in vitro, a composition comprising amounts of nucleic acid comprising polynucleotides encoding a desired trait. In addition, the composition comprises, for example, a relevant controlling promoter region made up of nucleotide sequences. This is combined with, for example, a gene delivery system comprising a cell transfection promotion agent such as retro viral vectors, adenoviral and adenoviral related vectors, or liposomal reagents or other agents used for gene therapy. These introduced under conditions effective to deliver the nucleic acid segments to the animal's germ cells optionally with the polynucleotide inserted into the genome of the germ cells. Following incorporation of the DNA, the treated animal is either allowed to breed naturally, or reproduced with the aid of assisted reproductive technologies, and the progeny selected for the desired trait.

This technology is applicable to the production of transgenic animals for use as animal models, and to the modification of the genome of an animal, including a human, by addition, modification, or subtraction of genetic material, often resulting in phenotypic changes. The present methods are also applicable to altering the carrier status of an animal, including a human, where that individual is carrying a gene for a recessive or dominant gene disorder, or where the individual is prone to pass a multigenic disorder to his offspring.

A preparation suitable for use with the present methods comprises a polynucleotide segment encoding a desired trait and a transfection promotion agent, and optionally an uptake promotion agent which is sometime equipped with agents protective against DNA breakdown. The different components of the transfection composition (mixture) are also provided in the form of a kit, with the components described above in measured form in two or more separate containers. The kit generally contains the different components in separate containers and instructions for effective use. Other components may also be provided in the kit as well as a carrier.

Thus the present technology is of great value in the study of stem cells and cellular development, and in producing transgenic vertebrate animals as well as for repairing genetic defects. The present technology is also suitable for germ line and stem cell line gene therapy in humans and other vertebrate animal species. The present invention is also valuable in identifying cell lineages before full differentiation to facilitate modification and/or engineering of specific tissues in vitro for their subsequent transplantation in the treatment of disease or trauma.

### BRIEF DESCRIPTION OF THE DRAWINGS

FIG. 1 represents a map of DNA construct pCyclinA1-EGFP-1.

FIG. 2 represents transcriptional start sites in the human cyclin A1 gene.

FIG. 3 represents 5′ upstream region of the human cyclin A1 gene.

FIG. 4 represents transactivation activity of cyclin A1 promoter fragments in Hela cells.

FIG. 5 shows activity of the cyclin A1 promoter fragments in the Drosophila cell line S2.

FIG. 6 shows effects of GC box (Sp1 site) mutations on promoter activity.

FIG. 7 shows cell cycle regulated activity of the cyclin A1 promoter in Hela cells.

FIG. 8 shows germ line-specific expression of EGFP from a human cyclin A1 promoter in murine testicular tissue.

FIG. 9 shows the positive association of cyclin A1 promoter methylation with silencing of a cyclin A1 promoter—EGFP transgene in MG63 cells and the repression of cyclin A1 promoter activity by methylation and MeCP2 in S2 Drosophila cells.

FIG. 10 shows a comparison of reporter gene expression from different promoters, including the cyclin A1 promoter, in cell lines from various tissues.

FIG. 11 shows transactivation of the cyclin A1 promoter by c-myb.

### DETAILED DESCRIPTION OF THE PREFERRED EMBODIMENTS

The present invention arose from a desire by the present inventors to improve on existing methods for the genetic modification of an animal's germ cells and for producing transgenic animals. The pre-existing art methods rely on direct injection of DNA into zygotes produced in vitro or in vivo, or by the production of chimeric embryos using embryonal stem cells incorporated into a recipient blastocyst. Following this, such treated embryos are transferred to the primed uterus or oviduct. The available methods are extremely slow and costly, rely on several invasive steps, and only produce transgenic progeny sporadically and unpredictably.

In their search for a less costly, faster, and more efficient approach for producing transgenics, the present inventors devised the present method which relies on the in vivo or ex vivo (in vitro) transfection of male animal germ cells with a nucleic acid segment encoding a desired trait. The present method relies on at least one of the following strategies. A first method delivers the nucleic acid segment using known gene delivery systems in situ to the gonad of the animal (in vivo transfection), allows the transfected germ cells to differentiate in their own milieu, and then selects for animals exhibiting the nucleic acid's integration into its germ cells (transgenic animals). The thus selected animals may be mated, or their sperm utilized for insemination or in vitro fertilization to produce transgenic progeny. The selection may take place after biopsy of one or both gonads, or after examination of the animal's ejaculate amplified by the polymerase chain reaction to confirm the incorporation of the desired nucleic acid sequence. In order to simplify the confirmation of the actual incorporation of the desired nucleic acid, the initial transfection may include a co-transfected reporter gene, such as a gene encoding for Green Fluorescent Protein (or encoding enhanced Green Fluorescent Protein [EGFP]), Yellow Fluorescent Protein, Blue Fluorescent Protein, a phycobiliprotein, such as phycoerythrin or phycocyanin, or any other protein which fluoresces under a suitable wave-length of ultraviolet light.

Alternatively, male germ cells may be isolated from a donor animal and transfected, or genetically altered in vitro to impart the desired trait. Following this genetic manipulation, germ cells which exhibit any evidence that the DNA has been modified in the desired manner are selected, and transferred to the testis of a suitable recipient animal. Further selection may be attempted after biopsy of one or both gonads, or after examination of the animal's ejaculate amplified by the polymerase chain reaction to confirm whether the desired nucleic acid sequence was actually incorporated. As described above, the initial transfection may have included a co-transfected reporter gene, such as a gene encoding the Green Fluorescent Protein (or enhanced Green Fluorescent Protein [EGFP]), Yellow Fluorescent Protein, Blue Fluorescent Protein, a phycobiliprotein, such as phycoerythrin or phycocyanin, or any other protein which fluoresces under light of suitable wave-lengths. Before transfer of the germ cells, the recipient testis are generally treated in one, or a combination, of a number of ways to inactivate or destroy endogenous germ cells, including by gamma irradiation, by chemical treatment, by means of infectious agents such as viruses, or by autoimmune depletion or by combinations thereof. This treatment facilitates the colonization of the recipient testis by the altered donor cells.

Animals that were shown to carry suitably modified sperm cells then may be either allowed to mate naturally, or alternatively their spermatozoa are used for insemination or in vitro fertilization. The thus obtained transgenic progeny may be bred, whether by natural mating or artificial insemination, to obtain further transgenic progeny. The method of this invention has a lesser number of invasive procedures than other available methods, and a high rate of success in producing incorporation into the progeny's genome of the nucleic acid sequence encoding a desired trait.

Primordial germ cells are thought to arise from the embryonic ectoderm, and are first seen in the epithelium of the endodermal yolk sac at the E8 stage. From there they migrate through the hindgut endoderm to the genital ridges. The primitive spermatogonial stem cells, known as A0/As, differentiate into type B spermatogonia. The latter further differentiate to form primary spermatocytes, and enter a prolonged meiotic prophase during which homologous chromosomes pair and recombine. Several morphological stages of meiosis are distinguishable: preleptotene, leptotene, zygotene, pachytene, secondary spermatocytes, and the haploid spermatids. The latter undergo further morphological changes during spermatogenesis, including the reshaping of their nucleus, the formation of acrosome, and assembly of the tail. The final changes in the spermatozoon take place in the genital tract of the female, prior to fertilization. The uptake of the nucleic acid segment administered by the present in vivo method to the gonads will reach germ cells that are at one or more of these stages, and be taken up by those that are at a more receptive stage. In the ex vivo (in vitro) method of genetic modification, generally only diploid spermatogonia are used for nucleic acid modification. The cells may be modified in vivo using gene therapy techniques, or in vitro using a number of different transfection strategies.

The inventors are, thus, providing in this patent a novel and unobvious method for isolation of male germ cells, and for the in vivo and ex vivo (in vitro) transfection of allogeneic as well as xenogeneic DNA into an animal's germ cells. This comprises the administration to an animal of a composition comprising a gene delivery system and at least one nucleic acid segment, in amounts and under conditions effective to modify the animal's germ cells, and allowing the nucleic acid segment to enter, and be released into, the germ cells, and to integrate into their genome.

The in vivo introduction of the gene delivery mixture to the germ cells may be accomplished by direct delivery into the animal's testis (es), where it is distributed to male germ cells at various stages of development. The in vivo method utilizes novel technology, such as injecting the gene delivery mixture either into the vasa efferentia, directly into the seminiferous tubules, or into the rete testis using, for example, a micropipette. To ensure a steady infusion of the gene delivery mixture, under pressures which will not damage the delicate tubule system in the testis, the injection may be made through the micropipette with the aid of a picopump delivering a precise measured volume under controlled amounts of pressure. The micropipette may be made of a suitable material, such as metal or glass, and is usually made from glass tubing which has been drawn to a fine bore at its working tip, e.g. using a pipette puller. The tip may be angulated in a convenient manner to facilitate its entry into the testicular tubule system. The micropipette may be also provided with a beveled working end to allow a better and less damaging penetration of the fine tubules at the injection site. This bevel may be produced by means of a specially manufactured grinding apparatus. The diameter of the tip of the pipette for the in vivo method of injection may be about 15 to 45 microns, although other sizes may be utilized as needed, depending on the animal's size. The tip of the pipette may be introduced into the rete testis or the tubule system of the testicle, with the aid of a binocular microscope with coaxial illumination, with care taken not to damage the wall of the tubule opposite the injection point, and keeping trauma to a minimum. On average, a magnification of about ×25 to ×80 is suitable, and bench mounted micromanipulators are not severally required as the procedure may be carried out by a skilled artisan without additional aids. A small amount of a suitable, non-toxic dye, may be added to the gene delivery fluid to confirm delivery and dissemination to the tubules of the testis. It may include a dilute solution of a suitable, non-toxic dye, which may be visualized and tracked under the microscope.

In this manner, the gene delivery mixture is brought into intimate contact with the germ cells. The gene delivery mixture typically comprises the modified nucleic acid encoding the desired trait, together with a suitable promoter sequence, and optionally agents which increase the uptake of the nucleic acid sequence, such as liposomes, retroviral vectors, adenoviral vectors, adenovirus enhanced gene delivery systems, or combinations thereof A reporter construct such as the gene encoding for Green Fluorescent Protein may further be added to the gene delivery mixture. Targeting molecules such as c-kit ligand may be added to the gene delivery mixture to enhance the transfer of the male germ cell.

For the ex vivo (in vitro) method of genetic alteration, the introduction of the modified germ cells into the recipient testis may be accomplished by direct injection using a suitable micropipette. Support cells, such as Leydig or Sertoli cells that provide hormonal stimulus to spermatogonial differentiation, may be transferred to a recipient testis along with the modified germ cells. These transferred support cells may be unmodified, or, alternatively, may themselves have been transfected, together with- or separately from the germ cells. These transferred support cells may be autologous or heterologous to either the donor or recipient testis. A preferred concentration of cells in the transfer fluid may easily be established by simple experimentation, but will likely be within the range of about 1×10⁵-10×10⁵ cells per 10 μl of fluid. This micropipette may be introduced into the vasa efferentia, the rete testis or the seminiferous tubules, optionally with the aid of a picopump to control pressure and/or volume, or this delivery may be done manually. The micropipette employed is in most respects similar to that used for the in vivo injection, except that its tip diameter generally will be about 70 microns. The microsurgical method of introduction is similar in all respects to that used for the in vivo method described above. A suitable dyestuff may also be incorporated into the carrier fluid for easy identification of satisfactory delivery of the transfected germ cells.

Once in contact with germ cells, whether they are in situ in the animal or vitro, the gene delivery mixture facilitates the uptake and transport of the xenogeneic genetic material into the appropriate cell location for integration into the genome and expression. A number of known gene delivery methods may be used for the uptake of nucleic acid sequences into the cell.

“Gene delivery (or transfection) mixture”, in the context of this patent, means selected genetic material together with an appropriate vector mixed, for example, with an effective amount of lipid transfecting agent. The amount of each component of the mixture is chosen so that the transfection of a specific species of germ cell is optimized. Such optimization requires no more than routine experimentation. The ratio of DNA to lipid is broad, preferably about 1:1, although other proportions may also be utilized depending on the type of lipid agent and the DNA utilized. This proportion is not crucial.

“Transfecting agent”, as utilized herein, means a composition of matter added to the genetic material for enhancing the uptake of exogenous DNA segment(s) into a eukaryotic cell, preferably a mammalian cell, and more preferably a mammalian germ cell. The enhancement is measured relative to the uptake in the absence of the transfecting agent. Examples of transfecting agents include adenovirus-transferrin-polylysine-DNA complexes. These complexes generally augment the uptake of DNA into the cell and reduce its breakdown during its passage through the cytoplasm to the nucleus of the cell. These complexes may be targeted to the male germ cells using specific ligands which are recognized by receptors on the cell surface of the germ cell, such as the c-kit ligand or modifications thereof.

Other preferred transfecting agents include lipofectin, lipfectamine, DIMRIE C, Superfect, and Effectin (Qiagen). Although these are not as efficient transfecting agents as viral transfecting agents, they have the advantage that they facilitate stable integration of xenogeneic DNA sequence into the vertebrate genome, without size restrictions commonly associated with virus-derived transfecting agents.

“Virus”, as used herein, means any virus, or transfecting fragment thereof, which may facilitate the delivery of the genetic material into male germ cells. Examples of viruses which are suitable for use herein are adenoviruses, adeno-associated viruses, retroviruses such as human immune-deficiency virus, lentiviruses, such as Moloney murine leukemia virus and the retrovirus vector derived from Moloney virus called vesicular-stomatitis-virus-glycoprotein (VSV-G)-Moloney murine leukemia virus, mumps virus, and transfecting fragments of any of these viruses, and other viral DNA segments that facilitate the uptake of the desired DNA segment by, and release into, the cytoplasm of germ cells and mixtures thereof. The mumps virus is particularly suited because of its affinity for immature sperm cells including spermatogonia. All of the above viruses may require modification to render them non-pathogenic or less antigenic. Other known vector systems, however, may also be utilized within the confines of the invention.

“Genetic material”, as used herein, means DNA sequences capable of imparting novel genetic modification(s), or biologically functional characteristic(s) to the recipient animal. The novel genetic modification(s) or characteristic(s) may be encoded by one or more genes or gene segments, or may be caused by removal or mutation of one or more genes, and may additionally contain regulatory sequences. The transfected genetic material is preferably functional, that is it expresses a desired trait by means of a product or by suppressing the production of another. Examples of other mechanisms by which a gene's function may be expressed are genomic imprinting, i.e. inactivation of one of a pair of genes (alleles) during very early embryonic development, or inactivation of genetic material by mutation or deletion of gene sequences, or by repression of a dominant negative gene product, among others.

In addition, novel genetic modification(s) may be artificially induced mutations or variations, or natural allelic mutations or variations of a gene(s). Mutations or variations may be induced artificially by a number of techniques, all of which are well known in the art, including chemical treatment, gamma irradiation treatment, ultraviolet radiation treatment, ultraviolet radiation, and the like. Chemicals useful for the induction of mutations or variations include carcinogens such as ethidium bromide and others known in the art.

DNA segments of specific sequences may also be constructed to thereby incorporate any desired mutation or variation or to disrupt a gene or to alter genomic DNA. Those skilled in the art will readily appreciate that the genetic material is inheritable and is, therefore, present in almost every cell of future generations of the progeny, including the germ cells.

Among novel characteristics are the expression of a previously unexpressed trait, augmentation or reduction of an expressed trait, over expression or under expression of a trait, ectopic expression, that is expression of a trait in tissues where it normally would not be expressed, or the attenuation or elimination of a previously expressed trait. Other novel characteristics include the qualitative change of an expressed trait, for example, to palliate or alleviate, or otherwise prevent expression of an inheritable disorder with a multigenic basis.

For the expression of transfected genetic material to obtain a desired trait, a promoter sequence is operably linked to a polynucleotide sequence encoding the desired trait or product. A promoter sequence is chosen that operates in the cell type of interest.

A promoter sequence, which is only active in cycling spermatogonial stem cell populations can be used for differential expression in male germ cells, for example, B-Myb or a spermotogonia specific promoter, such as the c-kit promoter region, c-raf-1 promoter, ATM (ataxia-telangiectasia) promoter, RBM (ribosome binding motif) promoter, DAZ (deleted in azoospermia) promoter, XRCC-1 promoter, HSP 90 (heat shock gene) promoter, or FRMI (from fragile X site) promoter.

The human cyclin A1 promoter region is a most preferred promoter sequence for driving the expression of a reporter construct or for driving the expression of another desired xenogeneic gene sequence, when expression is desired in germ cells, hematopoietic cells, other stem cells of a vertebrate.

The following nucleotide sequence represents the 5′ end of the human cyclin A1 gene. An untranscribed region extends from nucleotide −1299 to −1; a transcribed but untranslated region extends from +1 to +127, where the first ATG sequence begins; also represented are cyclin A1 exon 1 (+1 to +234), intron 1 (+235 to +537), and part of exon 2 (beginning at +538), with transcribed regions being underlined and the translational start site at nt. +127 to ++129 being bolded:

| | | |
|---|---|---|
| −1299 | TCGATCTGAT TTAGAGATTT AGGGATGGAT GTTTTAAAAA AAGCAAAAGT | (SEQ. ID. NO.:1). |
| −1249 | AGTAACAGAC TATAGCATTG GTAATGTGTG TGTGCATATA TACATATTAT | |
| −1199 | TTTTAAAAAA ATAAAGTTCG ATTATTTCAC CTGGCTTGTC AGTCACCTAT | |
| −1149 | GCAGGCGTCT GAGCCCCCGG GTTTCCAGGA GCCCCCCGTA TAAGGACCCC | |
| −1099 | AGGGACTCCT CTCCCCACGC GGCCGQGCCG CCCGCCCGGC CCCCAGCCCG | |
| −1049 | GAGAGCTGCC ACCGACCCCC TCAACGTCCC AAGCCCCAGC TCTGTCGCCC | |
| −0999 | GCGTTCCTTC CTCTTCCTGG GCCACAATCT TGGCTTTCCC GGGCCGGCTT | |
| −0949 | CACGCAGTTG CGCAGGAGCC CGCGGGGGAA GACCTCTCGT GGGGACCTCG | |
| −0899 | AGCACGACGT GCGACCCTAA ATCCCCACAT CTCGTCTGCC GCCTCGCAGG | |
| −0849 | CCACATGCAC CGGGAGCCGG GCGGGGCAGG CGCGGCCCGC AAGGACCCCC | |
| −0799 | GCGATGGAGA CGCAACACTG CCGCGACTGC ACTTGGGGCA GCCCCGCCGC | |
| −0749 | GTCCCAGCCG CCTCCCGGCA GGAAGCGTAG GTGTGTGAGC CGACCCGGAG | |
| −0699 | CGAGCCGCGC CCTCGGGCCA GCGTGGGCAG GGCGCCGCAG CCTGCGCAGC | |
| −0649 | CCCGAGGACC CCGCGTCGCT CTCCCGAGCC AGGGTTCTCA GGAGCGGGCC | |
| −0599 | GCGCAGGAGA CGTTAGAGGG GGTTGTTAGC GGCTGTTGGG AGAACGGGTC | |
| −0549 | ACGGAAACAG TCCCTTCCAA AGCCGGGGCC ATCGTGGGGT GGGCGAGTCC | |
| −0499 | GCCCTCCCAG GCCGGGGGCG CGGACCAGAG GGCACGTGTG CAGACGGCCG | |
| −0449 | CGGTCAGCCC CACCTCGCCC GGGCGGAGAC GCACAGCTGG AGCTGGAGGG | |
| −0399 | CCGTCGCCCG TTGGGCCCTC AGGGGCCTGA ACGCCCAGGG GTCGCGGCGA | |
| −0349 | GTCCACCCGG AGCGAGTCAG GTGAGCAGGT CGCCATGGCG ATGCGGCCCC | |
| −0299 | GGAGAGCGCA CGCCTGCCGC GGTCGGCATG GAAACGCTCC CGCTAGGTCC | |
| −0249 | GGGGGCGCCG CTGATTGGCC GATTCAACAG ACGCGGGTGG GCAGCTCAGC | |
| −0199 | CGCATCGCTA AGCCCGGCCG CCTCCCAGGC TGGAATCCCT CGACACTTGG | |
| −0149 | TCCTTCCCGC CCCGCCCTTC CGTGCCCTGC CCTTCCCTGC CCTTCCCCGC | |
| −0099 | CCTGCCCCGC CCGGCCCGGC CCGGCCCTGC CCAACCCTGC CCCGCCCTGC | |
| −0049 | CCCGCCCAGC CGGCCACCTC TTAACCGCGA TCCTCCAGTG CACTTGCCAG | |
| +0003 | TTGTTCCGGA CACATAGAAA GATAACGACG GGAAGACGGG GCCCCGTTTG | |
| +0053 | GGGTCCAGGC AGGTTTTGGG GCCTCCTGTC TGGTGGGAGG AGGCCGCAGC | |
| +0103 | GCAGCACCCT GCTCGTCACT TGGGATGGAG ACCGGCTTTC CCGCAATCAT | |
| +0153 | GTACCCTGGA TCTTTTATTG GGGGCTGGGG AGAAGAGTAT CTCAGCTGGG | |
| +0203 | AAGGACCGGG GCTCCCAGAT TTCGTCTTCC AGGTAACGTG GGTTTAGTAT | |
| +0253 | CCCGACTTGG AGGCTTGTCA GAATGTTTCT CTCCTTCCAG CCCAACACGA | |
| +0303 | AGTCTTGGGA TAAAAAGCCT CCCTCAGGGA TTCAAATAAC TGTTTTGATT | |
| +0353 | CAGAGCAACT TTGATCGCCT GTGCGGTCGC ACCTGCCCTT TCAGCCCCAA | |
| +0403 | TAATTACTGG GAAGATCAGC AATTGGTGTT AGTCCCATTG CTTGGTGCTC | |
| +0453 | TCCCTCCTAG AGGTTCGCTG TGTCCTTGGA GCCCGGGGTG GACGGAATCG | |
| +0503 | ACTAAACAGC TTGTCTGTTT CTCTTTCCCT GGTAGCAGCA GCCCGTGGAG | |
| +0553 | TCTGAAGCAA TGCACTGCAG CAACCCCAAG AGTGGAGTFG TGCTGGCTAC | |
| +0603 | AGTGGCCCGA GGTCCCGATG CTTGTCAGAT ACTCACCAGA GCCCCGCTGG | |
| +0653 | GCCAGGAT | |

A most preferred embodiment of the cyclin A1 promoter of the present invention is a DNA fragment with the sequence of nt. −1299 to +144, inclusive, having the first translational start site (the ATG in bold at nt. +127 to +129 of the human sequence above) changed to ATT (SEQ. ID. NO.2). Other preferred embodiments of a cyclin A1 promoter include any operative fragment of SEQ. ID. NO.:2 or non-human homologue thereof, or an operative derivative of any of these. Preferred examples of an operative fragment include the −1151 to +144 fragment (SEQ. ID. NO.:3), the −454 to +144 fragment (SEQ. ID. NO.:4), the −326 to +144 fragment (SEQ. ID. NO.:5), the −190 to +144 fragment (SEQ. ID. NO.:6), the −160 to +144 fragment (SEQ. ID. NO.:7), the −120 to +144 fragment (SEQ. ID. NO.:8), the −112 to +144 fragment (SEQ. ID. NO.:9), all with ATG at +127 to +129 changed as described above. But any cyclin A1 promoter fragment that includes the nucleotide sequence extending from nt. −112 downstream to at least nt. +5 or beyond, up to and including nt. +144, is also operative and useful, as long as the translational start site at +127 to +129 is no longer intact and the essential Sp1 binding sites between −112 and −37 (Gc Box Nos. 1, 2, and 3 and/or 4) are intact, as described below. Other preferred fragments, in accordance with the present invention, include those extending from −190 to +20 (SEQ. ID. NO.: 10), or from +190 to any nucleotide between nt. +20 up to nt. +144 (without the translational start site). But shorter fragments such as −190 to +13 (SEQ. ID. NO.:11), −190 to +6 (SEQ. I.D. NO.:12), or −190 to +5 (SEQ. ID. NO.:13) are also operative and useful. Non-human homologues include any cyclin A1 promoter sequence of non-human origin that functions in a vertebrate stem cell type of interest.

Another preferred embodiment of a cyclin A1 promoter is an operative derivative of SEQ. ID. NO:2, or of any operative fragment of SEQ. ID. NO. :2 or non-human homologue thereof, in which the codon of the first translational start site is changed to another codon sequence, other than ATT, that is also not recognized as a translational start site; another preferred cyclin A1 promoter is a derivative of SEQ. ID. NO. :2 with the codon of the first translational start site deleted altogether. Other operative derivatives include cyclin A1 promoter sequences containing a mutation, polymorphism, or variant allele with respect to any nucleotide position of SEQ. ID. NO.:2 that does not eliminate promoter activity.

Similar to promoters in other cell cycle regulatory genes (B. Henglein et al., Proc. Natl. Acad. Sci. (USA) 91:5490-94 [1994]; A. Hwang et al., J. Biol. Chem. 270:28419-24 [1995]; E. W. Lam et al., Oncogene 7:1885-90 [1992]), the cyclin A1 promoter does not possess a TATA-box motif. The nucleotides surrounding the transcriptional start site are likely to function as an initiator. The cyclin A1 promoter region contains multiple binding sites for transcription factor including GC boxes, Myb, and E2F sites.

The upstream region contains a GC rich region with multiple Sp1 binding sites that are essential for transcription from the cyclin A1 promoter. In contrast, predicted GC boxes in the cyclin A2 promoter are located more than 120 bp upstream of the transcriptional start site and these have not been shown to be essential for gene expression. GC boxes and the Sp 1 family transcription factors are important in the regulation of expression from the cyclin A1 promoter. Six GC boxes are found in the first 200 bp upstream of the transcription start site. Omitting the four GC boxes between −112 and −37 almost completely abrogates promoter activity. Among GC boxes Nos. 1-4, the two closest to the transcriptional start sites are most critical. Of GC boxes Nos. 3 and 4, only one of these is necessary for a basal level of transcriptional activity of the promoter.

Sp1, the main activating factor of the Sp1 family, and Sp3 can bind to GC boxes Nos. 1+2 and 3+4. Analysis of these fragments in insect cells demonstrates that Sp1 reconstitutes cyclin A1 promoter activity in all fragments that involve the GC boxes Nos. 1-4. Sp1 (or at least a member of the Sp1 family) is required for cyclin A1 promoter activity through interaction with elements located between −112 and −37. Repression is likely to be accomplished by Sp3 and an as yet unidentified repressor mechanism that does not depend on E2F, CDE or CHR elements.

The DNA of animal cells is subject to methylation at the 5′ carbon position of the cytidine bases of CpG dinucleotides. Unmethylated CpGs are found preferentially in transcriptionally active chromatin. (T. Naveh-Many et al., Active gene sequences are undermethylated, Proc. Natl. Acad. Sci. USA 78:4246-50 [1981]). Hypermethylation is associated with transcriptional repression. (R. Holliday, The inheritance of epigenetic defects, Science 238:163-70 [1987]).

Tissue-specific expression from the cyclin A1 promoter in male germ cells is seen irrespective of promoter methylation status. Even high levels of methylation do not inhibit cyclin A1 promoter expression during spermatogenesis. In contrast, expression from the cyclin A1 promoter in somatic tissues has been observed only in a transgenic mouse line that does not methylate the cyclin A1 promoter. This is evidence that the effects of methylation on gene expression are tissue-specific and can differ between somatic and germ cells.

High in vivo expression levels of cyclin A1 in mice and healthy humans are restricted to germ cells. (R. Yang etal. [1997]; Sweeney et al. [1996]). For an unknown reason, cyclin A1 is also frequently expressed at high levels in acute myeloid leukemia (R. Yang et al. [1997]; R. Yang et al., Cyclin A1 expression in leukemia and normal hematopoietic cells. Blood 93:2067-74 [1999]). Chromatin structure and probably changes in the methylation pattern contribute to tissue-specific expression. The cyclin A1 promoter is highly GC rich and bears a CpG island that extends over several hundred base pairs and ends about 50 base pairs upstream of the main transcriptional start site. When the methylation pattern of the CpG dinucleotides in the critical parts of the promoter was analyzed using bisulfite sequencing, as described in Example 22 below, a high degree of CpG methylation was observed in somatic, adherent cell lines but not in cyclin A1-expressing leukemia cell lines. Hypomethylation in leukemic cell lines is clearly restricted to the CpG island since a CpG at +114 outside of the CpG island was found to be completely methylated in all cell lines tested.

Therefore, for the purposes of obtaining selectable transgenic stem cells in accordance with the present method, silencing of expression from the cyclin A1 promoter in stem cell types other than germ cells is preferably prevented by flanking the promoter sequence and the reporter gene with insulator elements. For example, by including double copies of the 1.2 kb chicken β-globin insulator element 5′ to the cyclin A1 promoter sequence and 3′ to the reporter protein gene in the present DNA construct, methylation will be substantially prevented at CG dinucleotide sites within the CpG island of the cyclin A1 promoter sequence and thus expression of the reporter gene occurs within stem cell types other than germ cells. (M. J. Pikaart et al., Loss of transcriptional activity of a transgene is accompanied by DNA methylation and histone deacetylation and is prevented by insulators, Genes Dev. 12:2852-62 [1998]; Chung et al., DNA sequence which acts as a chromatin insulator element to protect expressed genes from cis-acting regulatory sequences in mammalian cells, U.S. Pat. No. 5,610,053).

Alternatively, when the method of obtaining selectable transgenic stem cells is practiced to select stem cells grown in vitro, inhibitors of histone deacetylation and DNA methylation, such as trichostatin A or sodium butyrate, can be included in the culture medium to prevent silencing of reporter expression from the cyclin A1 promoter in a wide variety of cultured stem cells. (M. J. Pikaart et al. [1998]).

Suppression of methylation of the cyclin A1 promoter sequence can sometimes cause expression from a cyclin A1 promoter in kidney podocytes or in B-cells. Consequently, in applications in which selectable kidney stem cells are of interest, in accordance with the present method of obtaining selectable transgenic stem cells, fluorescent or luminescent podocytes that express a reporter gene from a cyclin A1 promoter are easily distinguished from fluorescing or light-emitting transgenic kidney stem cells by the distinct podocyte morphology (including protruding pedicels). In applications in which hematopoietic stem cells are of interest, fluorescent or luminescent B-cells are distinguished from transgenic hematopoietic stem cells by additionally using a B-cell-specific antibody conjugated to a fluorescent label that fluoresces or emits at a different wavelength from that of the reporter protein expressed as a result of cyclin A1-promoted transcription. For example, phycoerythricin-conjugated monoclonal antibodies against B-cell-specific surface epitopes can be applied to a cell population sample from bone marrow to distinguish B-cells from transgenic hematopoietic stem cells.

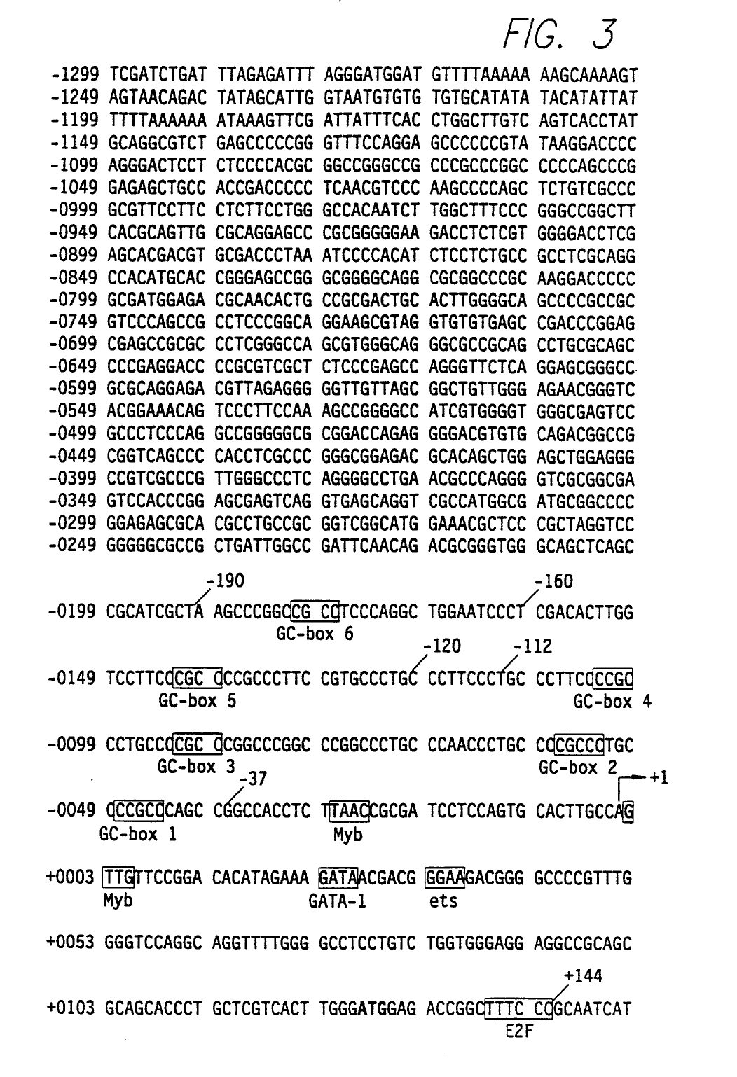

Three potential binding sites for Myb proteins are present within 100 bp of the transcription start sites of the cyclin A1 gene, located starting at −66, −27, and +2. (FIG. 3). Binding of c-myb protein occurs at the sites starting at −27 and +2, and c-myb protein transactivates expression from the human cyclin A1 promoter, as described in Example 23. In contrast, no consensus myb sites have been found for either the murine or human cyclin A2 promoter (X. Huet et aL, Molecular & Cellular Biology 16:3789-98 [1996]).

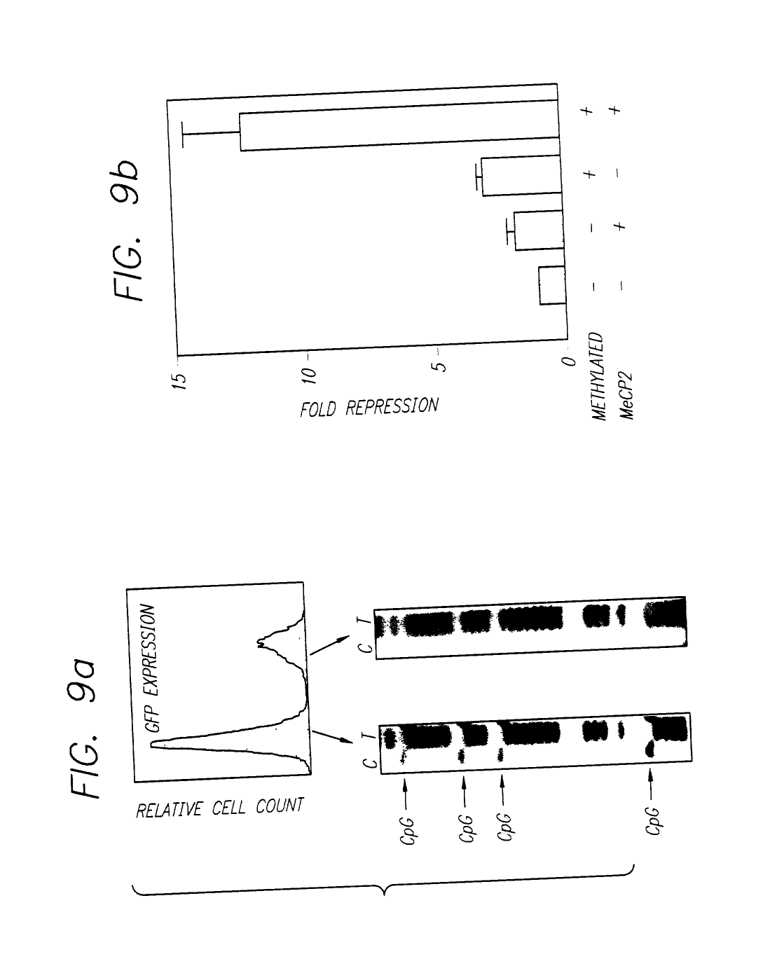

Similar to the cyclin A2 gene, two potential binding sites for transcription factor E2F are downstream of the transcriptional start site of cyclin A1. These E2F sites are not required for repression of cyclin A2 transcription in the G1 phase. (J. Zwicker et al. (1995) EMBO Journal 14, 4514-4522; X. Huet et al. [1996]). Likewise, the introduction of mutations in these sites in the cyclin A1 promoter does not alter the regulation of expression. Further evidence that these E2F sites are not relevant for regulation was shown using a 3 deletion (−190 to +13) that showed cell cycle regulation in vivo similar to the constructs containing both E2F sites (data not shown). Likewise, a 6-bp sequence that resembles the CDE of the human cyclin A2 gene was found in an antisense direction at position -19 to -24 (TCGCGG; SEQ. ID. NO.:32) of the cyclin A1 promoter. No significant differences in cell cycle regulation were found when these nucleotides were mutated (FIG. 9). This is consistent with the finding that these elements need to be in a 5′ to 3′ orientation to be functional (J. Zwicker et al. [1995]; N. Liu et al., Oncogene 16:2957-63 [1998]; N. Liu et al., Nucleic Acids Res. 25: 4915-20 [1997]).

The present invention relates to a method of obtaining a selectable transgenic stem cell from a vertebrate. The method involves transfecting a male germ cell or germ cell precursor with a transfection mixture, as described herein, that includes a polynucleotide, comprising a stem cell-specific promoter sequence, for example, a human or other homologous vertebrate cyclin A1 promoter sequence, or an operative fragment thereof, operatively linked to a gene encoding a fluorescent or light-emitting reporter protein, oriented so as to comprise a transcriptional unit. A polynucleotide containing the operatively linked stem cell-specific promoter and reporter gene, is incorporated in to the genome of a transfected male germ cell, or precursor, and can be transmitted to progeny after breeding, where it operates in stem cells of the progeny in vivo, such that in a cell population, taken from a progeny vertebrate's tissue or viewed in situ, stem cells differentially express the reporter gene compared to non-stem cells. Thus, these stem cells are readily selectable from the population of non-stem cells present in the tissue. Types of stem cells for which the method is useful include pluripotent, multipotent, bipotent, or monopotent stem cells, which includes male or female germ cells or stem cells related to any tissue of the vertebrate including, but not limited to, spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cells. And the present invention is also directed to a selectable transgenic stem cell, of any type, obtained by the method.

Preferred reporter genes encode fluorescent proteins including Green Fluorescent Protein (or EGFP), Yellow Fluorescent Protein, Blue Fluorescent Protein, a phycobiliprotein, such as phycoerythrin or phycocyanin, or any other protein which fluoresces under suitable wave-lengths of ultra-violet or other light. Another reporter gene suitable for some applications is a gene encoding a protein that can enzymatically lead to the emission of light from a substrate(s); for purposes of the present invention, such a protein is a “light-emitting protein.” For example, a light-emitting protein includes proteins such as luciferase or apoaequorin.

In particular applications involving a transfected cell that expresses additional xenogeneic genes from any promoter, this expression may be linked to a reporter gene that encodes a different fluorescent or light-emitting protein from the reporter gene linked to the cyclin A1 promoter. Thus, multiple reporters fluorescing or emitting at different wavelengths can be chosen and cell selections based on the expression of multiple traits can be made. The selectable transgenic stem cells may be sorted, isolated or selected from non-stem cells with the aid of, for example, a FACS scanner set at the appropriate wavelength(s). Alternatively, they are isolated or selected manually from non-stem cells using conventional microscopic technology. It is an advantage of the present method of obtaining selectable transgenic stem cells that it allows stem cells to be selected or isolated from non-embryonic tissue.

The invention also relates to a nucleic acid construct comprising a human cyclin A1 promoter sequence in accordance with the present invention, or an operative fragment thereof. In a preferred embodiment for use in the method of obtaining a selectable transgenic stem cell, the cyclin A1 promoter is operatively linked to a DNA having a nucleotide sequence encoding a fluorescent protein or a light emitting protein. Other preferred embodiments employ a xenogeneic nucleic acid encoding any desired product or trait. For purposes of the present invention, “operatively linked” means that the promoter sequence, is located directly upstream from the coding sequence and that both sequences are oriented in a 5′ to 3′ manner, such that transcription could take place in vitro in the presence of all essential enzymes, transcription factors, co-factors, activators, and reactants, under favorable physical conditions, e.g., suitable pH and temperature. This does not mean that, in any particular cell, conditions will favor transcription. For example, transcription from a cyclin A1 promoter is not favored in most differentiated cell types in transgenic animals.

The present invention also relates to a transgenic vertebrate cell containing the nucleic acid construct of the present invention, regardless of the method by which the construct was introduced into the cell. The present invention also relates to transgenic non-human vertebrates comprising such cells.

The present invention also relates to a kit for transfecting a male vertebrate's germ cells, which is useful for obtaining selectable transgenic stem cells. The kit is a ready assemblage of materials for facilitating the transfection of a vertebrate male germ cell. A kit of the present invention contains a transfecting agent, as described above, and a polynucleotide that includes a stem cell-specific promoter sequence operatively linked to a DNA sequence encoding a fluorescent or light-emitting protein, together with instructions for using the components effectively. Preferably, the kit includes a nucleic acid construct of the present invention. Optionally, the kit can include an immunosuppressing agent, such as cyclosporin or a corticosteroid, and/or an additional nucleotide sequence encoding for the expression of a desired trait. The materials or components assembled in the kit are provided to the practitioner stored in any convenient and suitable way that preserves their operability and utility. For example the components can be in dissolved, dehydrated, or lyophilized form; they can be provided at room, refrigerated or frozen temperatures.

This invention also relates to a method for the isolation of spermatogonia, comprising obtaining spermatogonia from a mixed population of testicular cells by extruding the cells from the seminiferous tubules and gentle enzymatic disaggregation. The spermatogonia or stem cells which are to be genetically modified, may be isolated from a mixed cell population by a novel method including the utilization of a promoter sequence, which is only active in stem cells, such as human cyclin A1 promoter, or in cycling spermatogonia stem cell populations, for example, B-Myb or a spermotogonia specific promoter, such as the c-kit promoter region, c-raf-1 promoter, ATM (ataxia-telangiectasia) promoter, RBM (ribosome binding motif) promoter, DAZ (deleted in azoospermia) promoter, XRCC-1 promoter, HSP 90 (heat shock gene) promoter, or FRMI (from fragile X site) promoter, linked to a reporter construct, for example, a construct comprising a gene encoding Green Fluorescent Protein (or EGFP), Yellow Fluorescent Protein, Blue Fluorescent Protein, a phycobiliprotein, such as phycoerythrin or phycocyanin, or any other protein which fluoresces under suitable wavelengths of light. These unique promoter sequences drive the expression of the reporter construct only in the cycling spermatogonia. The spermatogonia, thus, are the only cells in the mixed population which will express the reporter construct(s) and they, thus, may be isolated on this basis. In the case of a fluorescent reporter construct, the cells may be sorted with the aid of, for example, a FACS set at the appropriate wavelength(s) or they may be selected by chemical methods.

The method of the invention is suitable for application to a variety of vertebrate animals, all of which are capable of producing gametes, i.e. sperm or ova. Thus, in accordance with the invention novel genetic modification(s) and/or characteristic(s) may be imparted to animals, including mammals, such as humans, non-human primates, for example simians, marmosets, domestic agricultural (farm) animals such as sheep, cows, pigs, horses, particularly race horses, marine mammals, feral animals, felines, canines, pachyderms, rodents such as mice and rats, gerbils, hamsters, rabbits, and the like. Other animals include fowl such as chickens, turkeys, ducks, ostriches, emus, geese, guinea fowl, doves, quail, rare and ornamental birds, and the like. Of particular interest are endangered species of wild animal, such rhinoceros, tigers, cheetahs, certain species of condor, and the like.

The present invention is also related to a transgenic non-human vertebrate comprising a selectable transgenic stem cell in accordance with the present invention. Broadly speaking, a “transgenic” vertebrate animal is one that has had foreign DNA permanently introduced into its cells. The foreign gene(s) which (have) been introduced into the animal's cells is (are) called a “transgene(s)”. The present invention is applicable to the production of transgenic animals containing xenogeneic, i.e., exogenous, transgenic genetic material, or material from a different species, including biologically functional genetic material, in its native, undisturbed form in which it is present in the animal's germ cells. In other instances, the genetic material is “allogeneic” genetic material, obtained from different strains of the same species, for example, from animals having a “normal” form of a gene, or a desirable allele thereof. Also the gene may be a hybrid construct consisting of promoter DNA sequences and DNA coding sequences linked together. These sequences may be obtained from different species or DNA sequences from the same species that are not normally juxtaposed. The DNA construct may also contain DNA sequences from prokaryotic organisms, such as bacteria, or viruses.

In one preferred embodiment, the transfected germ cells of the transgenic animal have the non-endogenous (exogenous) genetic material integrated into their chromosomes. This is what is referred to as a “stable transfection”. This is applicable to all vertebrate animals, including humans. Those skilled in the art will readily appreciate that any desired traits generated as a result of changes to the genetic material of any transgenic animal produced by this invention are inheritable. Although the genetic material was originally inserted solely into the germ cells of a parent animal, it will ultimately be present in the germ cells of future progeny and subsequent generations thereof. The genetic material is also present in all other cells of the progeny, including somatic cells and all non-stem cells, of the progeny. This invention also encompasses progeny resulting from breeding of the present transgenic animals. The transgenic animals bred with other transgenic or non-transgenic animals of the same species will produce some transgenic progeny, which should be fertile. This invention, thus, provides animal line(s) which result from breeding of the transgenic animal(s) provided herein, as well as from breeding their fertile progeny.

“Breeding”, in the context of this patent, means the union of male and female gametes so that fertilization occurs. Such a union may be brought about by natural mating, i.e. copulation, or by in vitro or in vivo artificial means. Artificial means include, but are not limited to, artificial insemination, in vitro fertilization, cloning and embryo transfer, intracytoplasmic spermatozoal microinjection, cloning and embryo splitting, and the like. However, others may also be employed.

The transfection of mature male germ cells may be also attained utilizing the present technology upon isolation of the cells from a vertebrate, as is known in the art, and exemplified in Example 10. The thus isolated cells may then be transfected ex vivo (in vitro), or prepared for cryostorage, as described in Example 11. The actual transsection of the isolated testicular cells may be accomplished, for example, by isolation of a vertebrate's testes, decapsulation and teasing apart and mincing of the seminiferous tubules. The separated cells may then be incubated in an enzyme mixture comprising enzymes known for gently breaking up the tissue matrix and releasing undamaged cells such as, for example, pancreatic trypsin, collagenase type I, pancreatic DNAse type I, as well as bovine serum albumin and a modified DMEM medium. The cells may be incubated in the enzyme mixture for a period of about 5 min to about 30 min, more preferably about 15 to about 20 min, at a temperature of about 33° C. to about 37° C., more preferably about 36 to 37° C. After washing the cells free of the enzyme mixture, they may be placed in an incubation medium such as DMEM, and the like, and plated on a culture dish. Any of a number of commercially available transfection mixtures may be admixed with the polynucleotide encoding a desire trait or product for transfection of the cells. The transfection mixture may then be admixed with the cells and allowed to interact for a period of about 2 hrs to about 16 hrs, preferably about 3 to 4 hrs, at a temperature of about 33° C. to about 37° C., preferably about 36° C. to 37° C., and more preferably in a constant and/or controlled atmosphere. After this period, the cells are preferably placed at a lower temperature of about 33° C. to about 34° C., preferably about 30-35° C. for a period of about 4 hrs to about 20 hrs, preferably about 16 to 18 hrs. Other conditions which do not deviate radically from the ones described may also be utilized as an artisan would know.

The present method is applicable to the field of gene therapy, since it permits the introduction of genetic material encoding and regulating specific genetic traits. Thus, in the human, for example, by treating parents it is possible to correct many single gene disorders which otherwise might affect their children. It is similarly possible to alter the expression of fully inheritable disorders or those disorders having at least a partially inherited basis, which are caused by interaction of more than one gene, or those which are more prevalent because of the contribution of multiple genes. This technology may also be applied in a similar way to correct disorders in animals other than human primates. In some instances, it may be necessary to introduce one or more “gene(s)” into the germ cells of the animal to attain a desired therapeutic effect, as in the case where multiple genes are involved in the expression or suppression of a defined trait. In the human, examples of multigenic disorders include diabetes mellitus caused by deficient production of, or response to, insulin, inflammatory bowel disease, certain forms of atheromatus cardiovascular disease and hypertension, schizophrenia and some forms of chronic depressive disorders, among others. In some cases, one gene may encode an expressible product, whereas another gene encodes a regulatory function, as is known in the art. Other examples are those where homologous recombinant methods are applied to repair point mutations or deletions in the genome, inactivation of a gene causing pathogenesis or disease, or insertion of a gene that is expressed in a dominant negative manner, or alterations of regulating elements such as gene promoters, enhancers, the untranslated tail region of a gene, or regulation of expansion of repeated sequences of DNA which cause such diseases as Huntingdon's chorea, Fragile-X syndrome and the like.

A specific reproductive application of the present method is to the treatment of animals, particularly humans, with disorders of spermatogenesis. Defective spermatogenesis or spermiogenesis frequently has a genetic basis, that is, one or several mutations in the genome may result in failure of production of normal sperm cells. This may happen at various stages of the development of germ cells, and may result in male infertility or sterility. The present invention is applicable, for example, to the insertion or incorporation of nucleic acid sequences into a recipient's genome and, thereby, establish spermatogenesis in the correction of oligozoospermia or azoospermia in the treatment of infertility. Similarly, the present methods are also applicable to males whose subfertility or sterility is due to a motility disorder with a genetic basis.

The present method is additionally applicable to the generation of transgenic animals expressing agents which are of therapeutic benefit for use in human and veterinary medicine or well being. Examples include the production of pharmaceuticals in domestic cows' milk, such as factors which enhance blood clotting for patients with types of haemophilia, or hormonal agents such as insulin and other peptide hormones.

The present method is further applicable to the generation of transgenic animals of a suitable anatomical and physiological phenotype for human xenograft transplantation. Transgenic technology permits the generation of animals which are immune-compatible with a human recipient. Appropriate organs, for example, may be removed from such animals to allow the transplantation of, for example, the heart, lung and kidney.

In addition, germ cells transfected in accordance with this invention may be extracted from the transgenic animal, and stored under conditions effective for later use, as is known in the art. Storage conditions include the use of cryopreservation using programmed freezing methods and/or the use of cryoprotectants, and the use of storage in substances such as liquid nitrogen. The germ cells may be obtained in the form of a male animal's semen, or separated spermatozoa, or immature spermatocytes, or whole biopsies oftesticular tissue containing the primitive germ cells. Such storage techniques are particularly beneficial to young adult humans or children, undergoing oncological treatments for such diseases such as leukemia or Hodgkin's lymphoma. These treatments frequently irreversibly damage the testicle and, thus, render it unable to recommence spermatogenesis after therapy by, for example, irradiation or chemotherapy. The storage of germ cells and subsequent testicular transfer allows the restoration of fertility. In such circumstances, the transfer and manipulation of germ cells as taught in this invention are accomplished, but transfection is generally not relevant or needed.

In species other than humans, the present techniques are valuable for transport of gametes as frozen germ cells. Such transport will facilitate the establishment of various valued livestock or fowl, at a remote distance from the donor animal. This approach is also applicable to the preservation of endangered species across the globe.

The method of obtaining selectable transgenic stem cells, the selectable transgenic stem cells, the transgenic non-human vertebrates and vertebrate semen, and the nucleic acid contructs and kits, in accordance with the present invention, are valuable tools in the study of cellular differentiation and development and in developing new therapies for diseases related to cell differentiation, such as cancer, or for the regeneration of tissues after traumatic injuries. The present invention is valuable in identifying cell lineages before full differentiation to facilitate modification and/or engineering of specific tissues in vitro for their subsequent transplantation in the treatment of disease or trauma. It is an advantage of the present method of obtaining selectable transgenic stem cells that it allows stem cells to be selected or isolated from non-embryonic tissue, thus avoiding potential ethical and legal problems associated with the use of embryonic tissue. It is a further advantage that in accordance with the present invention, selectable transgenic stem cells can be selected and analyzed whether grown in vivo (i.e., in the whole organism) or in vitro.

The invention will now be described in greater detail by reference to the following non-limiting examples. The pertinent portions of the contents of all references, and published patent applications cited throughout this patent necessary for enablement purposes are hereby incorporated by reference.

### EXAMPLES

In Vivo and In Vitro Adenovirus-enhanced Transferrin-polylysine-mediated Delivery of Green Fluorescent Protein Reporter Gene to Testicular Cells and Expression

The adenovirus enhanced transferrin-polylysine-mediated gene delivery system has been described and patented by Curiel et al. (D. T. Curiel et al. Adenovirus enhancement of transferrin-polylysine-mediated gene delivery, PNAS USA 88: 8850-8854 (1991). The delivery of DNA depends upon endocytosis mediated by the transferrin receptor (Wagner et al., Transferrin-polycation conjugates as carriersfor DNA uptake into cells, Proc. Natl. Acad. Sci. (USA) 87:3410-3414 (1990). In addition this method relies on the capacity of adenoviruses to disrupt cell vesicles, such as endosomes and release the contents entrapped therein. This system can enhance the gene delivery to mammalian cells by as much as 2,000 fold over other methods.

The gene delivery system employed for the in vivo and in vitro experiments was prepared as shown in examples below.

### Example 1

Preparation of Transferrin-poly-L-Lysine Complexes

Human transferrin was conjugated to poly (L-lysine) using EDC (1-ethyl-3-(3-dimethyl aminopropyl carbodiimide hydrochloride) (Pierce), according to the method of Gabarek and Gergely (Gabarek & Gergely, Zero-length cross-linking procedure with the use of active esters, Analyt. Biochem 185 131 (1990)). In this reaction, EDC reacts with a carboxyl group of human transferrin to form an amine-reactive intermediate. The activated protein was allowed to react with the poly (L-lysine) moiety for 2 hrs at room temperature, and the reaction was quenched by adding hydroxylamine to a final concentration of 10 mM. The conjugate was purified by gel filtration, and stored at −20° C.

### Example 2

Preparation of DNA for In vivo Trasfection

The Green Lantern-1 vector (Life Technologies, Gibco BRL, Gaithersberg, Md.) is a reporter construct used for monitoring gene transfection in mammalian cells. It consists of the gene encoding the Green Fluorescent Protein (GFP) driven by the cytomegalovirus (CMV) immediate early promoter. Downstream of the gene is a SV40 polyadenylation signal. Cells transfected with Green Lantern-1 fluoresce with a bright green light when illuminated with blue light. The excitation peak is 490 nm.

### Example 3

Preparation of Adenoviral Particles

Adenovirus dI312, a replication-incompetent strain deleted in the E1a region, was propagated in the E1a trans-complementing cell line 293 as described by Jones and Shenk (Jones and Shenk, PNAS USA (1979) 79: 3665-3669). A large scale preparation of the virus was made using the method of Mittereder and Trapnell (Mittereder et al., “Evaluation of the concentration and bioactivity of adenovirus vectors for gene therapy”, J. Urology, 70: 7498-7509 (1996)). The virion concentration was determined by UV spectroscopy, 1 absorbance unit being equivalent to 10 viral particles /ml. The purified virus was stored at −70° C.

### Example 4

Formation of Transferrin-poly-L Lysine-DNA-Viral Complexes

6 μg transferrin-polylysine complex from Example 1 were mixed in 7.3×10⁷ adenovirus d1312 particles prepared as in Example 3, and then mixed with 5 ug of the Green Lantern DNA construct of Example 2, and allowed to stand at room temperature for 1 hour. About 100 ul of the mixture were drawn up into a micropipette, drawn on a pipette puller, and slightly bent on a microforge. The filled micropipette was then attached to a picopump (Eppendorf), and the DNA complexes were delivered under continuous pressure, in vivo to mice as described in Example 6.

Controls were run following the same procedure, but omitting the transferrin-poly-lysine-DNA-viral complexes from the administered mixture.

### Example 5

Comparison of Adenovirus-enhanced Transferrin-polylysine & Lipofectin Mediated Transfection Efficiency

The conjugated adenovirus particle complexed with DNA were tested on CHO cells in vitro prior to in vivo testing. For these experiments a luciferase reporter gene was used due to the ease of quantifying luciferase activity. The expression construct consists of a reporter gene encoding luciferase, is driven by the CMV promoter (Invitrogen, Carlsbad, Calif. 92008). CHO cells were grown in Dulbecco's modified Eagle's medium (DMEM) with 10% fetal calf serum. For gene transfer experiments CHO cells were seeded into 6 cm tissue culture plates and grown to about 50% confluency (5×10⁵ cells). Prior to transfection the medium was aspirated and replaced with serum free DMEM. Cells were either transfected with transferrin-polylysine-DNA complexes or with lipofectin DNA aggregates. For the transferrin-polylsine mediated DNA transfer, the DNA-adenovirus complexes were added to the cells at a concentration of 0.05-3.2×10⁴ adenovirus particles per cell. Plates were returned to the 5% CO₂ incubator for 1 hour at 37° C. After 1 hour 3 ml of complete media was added to the wells and the cells were allowed to incubate for 48 hours before harvesting. The cells were removed from the plate, counted and then lysed for measurement of luciferase activity.

For cells transfected by lipofectin, 1 μg of CMV-luciferase DNA was incubated with 17 μl of Lipofectin (Life Technologies). The DNA-lipofectin aggregates were added to the CHO cells and allowed to incubate at 37° C. at 5% CO₂ for 4 hours. Three mls of complete medium was added then to the cells and they were allowed to incubate for 48 hours. The cells were harvested, counted and lysed for luciferase activity. The luciferase activity was measured by a luminometer. The results obtained are shown in Table 1.

The data included in Table 1 below show that the adenovirus-enhanced transferrin-polylysine gene delivery system is 1,808 fold more efficient than lipofection for transfection of CHO cells.

TABLE 1
Comparison of Lipofection & Adenovirus Enhanced
Transferrin-polylysine Transfection of CHO Cells

| | | Luciferase |
|---|---|---|
| Sample | Treatment | Activity (RLU) |
| 1 | 1 × 10⁷ particles + 6 ug CMV-Luc | 486 |
| 2 | 2.5 × 10⁷ particles + 6 ug CMV-Luc | 1,201 |
| 3 | 5.0 × 10⁷ particles + 6 ug CMV-luc | 11,119 |
| 4 | 1 × 10⁹ particles + 6 ug CMV-Luc | 2,003,503 |
| 5 | Lipofection | 1,108 |
| 6 | Unmanipulated cells | 155 |

### Example 6

In Vivo Delivery of DNA to Animal's Germ Cells via Tranferrin-L-lysine-DNA-Viral Complexes

The CMV-EGFP (Gibco-BRL, Life Technologies, Gaithersburg, Md. 20884) DNA-transferrin-polylysine viral complexes, prepared as described in Example 4 above, were delivered into the seminiferous tubules of three (3)-week-old B6D2F1 male mice. The DNA delivery by transferrin receptor-mediated endocytosis is described by Schmidt et al. and Wagner et al. (Schmidtetal., Cell 4: 41-51 (1986); Wagner, E., et al. PNAS (1990), (USA) 81: 3410-3414 (1990)). In addition, this delivery system relies on the capacity of adenoviruses to disrupt cell vesicles, such as endosomes and release the contents entrapped therein. The transfection efficiency of this system is almost 2,000 fold higher than lipofection.

The male mice were anesthetized with 2% Avertin (100% Avertin comprises 10 g 2,2,2-tribromoethanol (Aldrich) and 10 ml t-amyl alcohol (Sigma), and a small incision made in their skin and body wall, on the ventral side of the body at the level of the hind leg. The animal's testis was pulled out through the opening by grasping at the testis fat pad with forceps, and the vas efferens tubules exposed and supported by a glass syringe. The EGFP DNA-transferrin-polylysine viral complexes were injected into a single vasa efferentia using a glass micropipette attached to a hand held glass syringe or a pressurized automatic pipettor (Eppendorf), and Trypan blue added to visualize the entry of the mixture into the seminiferous tubules. The testes were then placed back in the body cavity, the body wall was sutured, the skin closed with wound clips, and the animal allowed to recover on a warm pad.

### Example 7

Detection of DNA and Transcribed Message

Nine (9) days after delivery of the genetic material to the animals' testis, two of the animals were sacrificed, their testes removed, cut in half, and frozen in liquid nitrogen. The DNA from one half of the tissues, and the RNA from the other half of the tissues were extracted and analyzed.

(a) Detection of DNA

The presence of DNA encoding enhanced green fluorescent protein (EGFP DNA) in the extracts was tested 9 days after administration of the transfection mixture using the polymerase chain reaction, and EGFP specific oligonucleotides. EGFP DNA was present in the testes of the animals that had received the DNA complexes, but was absent from sham operated animals.

(b) Detection of RNA

The presence of CMV-EGFP mRNA was assayed in the testes of experimental animals as follows. RNA was extracted from injected, and non-injected testes, and the presence of the EGFP messages was detected using reverse transcriptase PCR (RTPCR) with EGFP specific primers. The EGFP message was present in the injected testes, but not in the control testes. Thus, the DNA detected above by PCR analysis is, in fact, episomal EGFP DNA, or EGFP DNA which has integrated into the chromosomes of the animal. The transfected gene was being expressed.

Northern blot analysis was also done to confirm transcription regulated by the human cyclin A1 promoter. Total RNA was prepared from tissues using a RNA tissue preparation kit (Qiagen). Polyadenylated RNA was prepared by passage over an oligo(dT)-cellulose column. The RNA is polyoxylated and applied to 1.5% Agarose gel. After electrophoresis the RNA is transferred to nitrocelullose paper and hybridized with a cyclin A1 cDNA riboprobe. After hybridization the membrane was washed twice with 1x SSC at 60° C. for 1 hour. The washed membrane was exposed to X-ray film.

### Example 8

Expression of Non-endogenous DNA

Two males, one having received an injection with the EGFP transfection mixture and a control to whom only surgery was administered, were sacrificed 4 days after injection, and their testes excised, and fixed in 4% paraformaldehyde for 18 hours at 4° C. The fixed testis was then placed in 30% sucrose in PBS with 2 mM MgCl₂ for 18 hours at 4° C., embedded in OCT frozen on dry ice, and sectioned. When the testes of both animals were examined with a confocal microscope with fluorescent light at a wavelength of 488 nM, bright fluorescence was detected in the tubules of the EGFP-injected mice, but not in the testes of the controls. Many cells within the seminferous tubules of the EGFP-injected mouse showed bright fluorescence, which evidences that they were expressing Green Fluorescent Protein.

### Example 9

Generation of Offspring from Normal Matings

EGFP-transfected males were mated with normal females. The females were allowed to complete gestation, and the pups to be born. The pups (F1 offspring or progeny) were screened for the presence of the novel genetic material(s).

### Example 10

In Vitro Transfection of Testicular Cells

Cells were isolated from the testes of three 10-day-old mice. The testes were decapsulated and the seminiferous tubules were teased apart and minced with sterile needles. The cells were incubated in enzyme mixture for 20 minutes at 37° C. The enzyme mixture was made up of 10 mg bovine serum albumin (embryo tested), 50 mg bovine pancreatic trypsin type III, Clostridium collagenase type I, 1 mg bovine pancreatic DNAse type I in 10 mls of modified HTF medium (Irvine Scientific, Irvine, Calif.). The enzymes were obtained from Sigma Company (St. Louis, Mo. 63178). After digestion, the cells were washed twice by centrifugation at 500 x g with HTF medium and resuspended in 250 μl HTF medium. The cells were counted, and 0.5×10⁶ cells were plated in a 60 mm culture dish in a total volume of 5 ml DMEM (Gibco-BRL, Life Technologies, Gaithesburg, Md. 20884). A transfection mixture was prepared by mixing 5 μg Green Lantern DNA (Gibco-BRL, Life Technologies, Gaithesburg, Md. 20884) with 20 μSuperfect (Qiagen, Santa Clarita, Calif. 91355) and 150 μl DMEM. The transfection mix was added to the cells and they were allowed to incubate for 3 hours at 37° C., 5% CO₂ The cells were transferred to a 33° C. incubator and incubated overnight.

The following morning the cells were assessed for transfection efficiency by counting the number of fluorescent cells. In this experiment the transfection efficiency was 90% (Figure not shown). The testicular cells transfected with Green Lantern viewed with Nomaski optics x20 show the same cells viewed with FITC. Nearly all the cells were fluorescent, which is confirmation of their successful transfection.

The cells were injected into the testis via the vasa efferentia using a micropipette. 3×10⁵ cells in a total volume of 50 μl were used for the injection. The cells were mixed with Trypan blue prior to the injection. Three adult mice were injected with transfected cells. The Balb/cByJ recipient mice had been irradiated 6 weeks prior to the injection with 800 Rads of gamma irradiation. One mouse became sick and was sacrificed 48 hours after the injection. The testes from this mouse were dissected, fixed and processed for histology.

The two remaining males were bred with normal females as shown. After 4 months pups were born. Litters are currently being screened for the integration of the transgene.

### Example 11

Preparation of a Cell Suspension from Testicular Tissue for Cryopreservation

A cell suspension was prepared from mice of different ages as described below.

| | |
|---|---|
| Group I: | 7-10 day olds |
| Group II: | 15-17 day olds |
| Group III: | 24-26 day olds |

The mice's testes were dissected, placed in phosphate buffered saline (PBS) decapsulated, and the seminiferous tubules were teased apart. Seminiferous tubules from groups I and II were transferred to HEPES buffered culture medium (D-MEM) (Gibco-BRL, Life Technologies, Gaithesburg, Md. 20884) containing 1 mg/ml Bovine serum albumin (BSA) (Sigma, St. Louis, Mo. 63178) and Collagenase Type I (Sigma) for the removal of interstitial cells. After a 10 minute incubation at 33° C., the tubules were lifted into fresh culture medium. This enzymatic digestion was not carried out on the testes from group I because of their fragility.

The tubules from group II and III mice or the whole tissue from group I mice were transferred to a Petri dish with culture medium and were cut into 0. 1-1 mm pieces using a sterile scalpel and needle. The minced tissue was centrifuged at 500 x g for 5 minutes and the pellet was resuspended in 1 ml of enzyme mix. The enzyme mix was made up in D-DMEM with HEPES (Gibco-BRL) and consisted of 1 mg/ml bovine serum albumin (BSA) (Sigma, embryo tested), 1 mg/ml collagenase I (Sigma) and 5 mg/ml bovine pancreatic trypsin (Sigma) and 0.1 mg/ml deoxyribonuclease I (DN-EP, Sigma). The tubules were incubated in enzyme mix for 30 minutes at 33° C. After the incubation, 1 ml of medium was added to the mix and the cells were centrifuged at 500 x g for 5 min. The cells were washed twice in medium by centrifugation and resuspension. After the final wash the cell pellet was resuspended in 250 μl of culture medium and counted.

### Example 12

Cloning of the Cyclin A1 Gene and Construction of DNA Constructs Containing Cyclin A1-luciferase

Cloning of the Genomic Fragment of the Human cyclin A1.

The cyclin A1 gene was cloned by screening a genomic Fix II lambda library made from placenta (Stratagene) using the cyclin A1 cDNA as a probe. (R. Yang et aL [1997]). Of the several phage clones obtained, one contained all the exons and included a 1.3 kb region upstream of the 5′ end of the cDNA. A 2.2 kb NotI-Bam HI fragment from the 5′ end of the gene was subcloned into the pRS316 cloning vector. The construct was further digested using Sma I; and three fragments were subcloned into PUC19. The fragments were sequenced in both directions using cycle sequencing and an automated sequencer (ABI373) or Sequenase 2.0 (Amersham). The positions and lengths of the introns were determined by PCR amplification of the entire cyclin A1 coding region with different primers. Subsequently, PCR products were either subcloned using pGEM-T-Easy (Promega) or directly sequenced using cycle sequencing. Boundaries of the ˜4.5 kb intron 2 were determined by direct sequencing of the lambda phage clone.

Generation of Cyclin A1-luciferase DNA Constructs.

The initial luciferase constructs were generated by PCR amplification of the pRS316 plasmid containing the 2.2 kb cyclin A1 fragment. A BgII site at the 5′ end and a Bam HI site at the 3′ end were introduced and the Pfu amplified fragment was cloned into the Bg/II site of PGL3-Basic. The +144 fragment was generated to include the potential E2F site starting at +139. (FIG. 3). The ATG in the primer (the initiating codon for cyclin A1 at nt. +127 to +129) was mutated to ATT to avoid the initiation of translation. All constructs were confirmed to have the correct sequence by DNA sequencing. The 5′ deletions were generated by exonuclease III treatment using Kpn I/Sac I digested PGL3-Basic containing the −1299 to +144 fragment and the Erase-a-base kit (Promega). The endpoints of the deletions were determined by sequencing. The −37 fragment was constructed by digesting the −190 to +144 containing PGL3-Basic with NaeI and Hind III and subsequent cloning of the 200 bp fragment into PGL3-Basic digested with Sma I and Hind III.

Cell Culture and Transfection.

Hela cells were cultured in DMEM medium supplemented with 10% fetal calf serum (FCS) containing 100 U/ml Penicillin and 100 μg/mL Streptomycin. For transfection, 5×10⁵ cells were seeded into 60 mm plates 16 hours before transfection. Transfection was carried out using lipofectAMINE (Gibco, Life Technology) according to the manufacturer's protocol. Two μg of luciferase reporter plasmid was transfected together with 300 ng of a CMV-β-gal expression vector used for standardization. Cells were harvested and assayed for luciferase and β-galactosidase activity after 48 hours. All experiments were carried out in duplicate and were independently performed at least 3 times. Data of luciferase assays are shown as mean ±SEM of three independent experiments unless stated otherwise. The Drosophila cell line S2 was obtained from ATCC and grown at room temperature in Schneider's insect cell medium (Gibco) supplemented with 10% FCS. Insect cells were transfected using Superfect (Qiagen). Briefly, 5×10⁵ cells were seeded into 6 well plates and the superfect-DNA mixture was added dropwise. One μg of the luciferase reporter was transfected with or without 100 ng of the Sp1 expression vector pAC-Sp1 which was a kind gift from Dr. E. Stanbridge (UC Irvine). Luciferase activity was analyzed after 48 hours. Luciferase values could not be standardized using P-galactosidase activity because the viral promoters in the available plasmids also depended strongly on Sp1 for adequate expression. All experiments were carried out in duplicate and independently performed at least 3 times.

Cell Cycle Dependent Promoter Activity.

Hela cells were transfected using lipofectAMINE as described above. After transfection, cells were cultured in 0.1% FCS containing medium. After 16 h, medium was exchanged and cells were synchronized essentially as described. (D. Carbonaro-Hall et al., Oncogene 8:1649-59 [1993]). Cells were arrested in G₁ by serum starvation (0.1% FCS), in early S phase by aphidicolin (2,μ/ml), and in S phase by aphidicolin treatment and release into fresh medium 6 hours before harvest. Cells were arrested in G₂/M phase by nocodazole (0.1 μg/ml). Appropriate synchronization was confirmed by DNA quantitation using flow cytometry and the experiments were performed at least three times. For the cell cycle release experiments, Hela cells were arrested using aphidicolin as described above but cells were harvested at the different time points. The time course experiments were independently performed two times.

RACE and Primer Extension.

The rapid amplification of 5′ cDNA ends (RACE) was performed using a 5′ RACE system (Gibco). The procedure was performed as suggested in the manufacturer's protocol using RNA of the myeloid leukemia cell lines ML1 and U937. RNA was reversed transcribed using the primer 5′-CCC TCT CAG AAC AGA CAT ACA (SEQ. ID. NO.: 14; positions +981 to +961 of the cDNA) and Superscript II reverse transcriptase (Gibco). Gene-specific cDNA was PCR-amplified using the gene-specific primer 5′-CTG ATC CAG AAT AAC ACC TGA (SEQ. ID. NO.: 15; positions +460 to +440 of the cDNA) and the universal 5′ RACE Abridged Anchor Primer 5′-GGC CAC GCG TCG ACT AGT ACG GGI IGG GII GGG IIG (SEQ. ID. NO.: 16; I=inosine). PCR-amplifications from both RNA samples yielded a single band of about 450 bp. The entire PCR product was phenol-chloroform extracted, precipitated using NH4+acetate and finally cloned into pGEM-T-Easy and sequenced.

The primer extension assay was carried out by reverse transcription of 10 μg RNA (U937) using a ³²P-labeled primer 5′-CTC CTC CCA CCA GAC AGG A (SEQ. ID. NO.: 17) corresponding to +95 to +76 on the cDNA. Hybridization was carried out overnight at 58° C. Superscript II was used for reverse transcription at 42° C. for 50 minutes. Extension products were resolved on a 8% sequencing gel with a sequencing reaction being run in parallel. As negative controls, we used 10 μg of t-RNA and a sample without RNA.

Electrophoretic Mobility Shift Assays.

Nuclear extracts from Hela cells were prepared as described (A. M. Chumakov et al., Oncogene 8:3005-11 [1993]). For gel retardation experiments, 1 ng of ³²P-labeled double stranded oligonucleotides containing either GC boxes 1+2 (5′-CCT GCC CCG CCC TGC CCC GCC CAG CC; SEQ. ID. NO.:18) or GC boxes 3+4 (5′-CCT TCC CCG CCC TGC CCC GCC CGG CCC; SEQ. ID. NO.: 19) were incubated for 20 min at room temperature with 5 μg of Hela nuclear extract. The final reaction contained: 10 mM Tris-HCL, pH 7.5, 5% glycerol, 1 mM MgCl₂, 0.5 mM EDTA, 0.5 mM DTT, 100 mM NaCl and 1 μug poly(dI-dC)-poly(dI-dC). For competition experiments, 100 ng of double stranded oligonucleotide containing either a Sp1 consensus site (5′-ATT CGA TCG GGG CGG GGC GAG C; SEQ. ID. NO.:20), the oligonucleotide used for gel retardation (see above) or a non-specific oligonucleotide (5′-GAG ACC GGC TCG AAC GCA ATC ATG T; SEQ. ID. NO. :21) were preincubated for 15 min at room temperature with the nuclear extracts before the addition of the labeled oligonucleotide. For supershift experiments, 2-3 μg of polyclonal antibody against Sp1 (Pep2, Santa Cruz) or Sp3 (D20, Santa Cruz) were preincubated with the nuclear extracts. Reactions were loaded on a 0.5x TBE /4% non-denaturing polyacrylamide gel and run for 2-3 h at 10 V/cm. Gels were dried and autoradiographed.

Site Directed Mutagenesis.

Site directed mutagenesis was performed according to the method from Deng and Nickoloff(W. P. Deng and J. A. Nickoloff, Analyt. Biochem.200:81-88 [1992]) using the Transformer site directed mutagenesis kit (Clontech). In brief, phosphorylated oligonucleotides containing the desired mutation were annealed on the single-stranded PGL3-Basic plasmid (containing the fragment −190 to +144) together with the oligonucleotide 5′-AAT CGA TAA GAA TTC GTC GAC CGA (SEQ. ID. NO.:22) that changes the unique Bam HI site to an Eco RI site. The complementary strand was extended and completed from the annealed oligos using T4 Polymerase and T4 Ligase. Selection for the mutant plasmid was performed by two rounds of digestion with Bam HI and subsequent transformations, first into the repair deficient strain BMH 71-18 muts and finally into DH5a. The entire promoter fragment was sequenced to verify desired mutations and to exclude second site mutations. Because of the short distances between GC boxes 1+2 and 3+4, oligonucleotides were designed to mutate both GC boxes simultaneously. Mutations in all 4 GC boxes were introduced by simultaneously adding oligos 1+2 and 3+4. All oligonucleotides used in these experiments were 5′-phosphorylated. The following oligonucleotides were used (mutated bases underlined):

| | | |
|---|---|---|
| GC box 1: | CCC CGC CCT GCC CCT TAC AGC CGG CCA CC | (SEQ. ID. NO.:23), |
| GC box 2: | CCA ACC CTG CCC TTA CCT GCC CCG | (SEQ. ID. NO.:24), |
| GC box 3: | CCC TGC CCC TTC CGG CCC GGC C | (SEQ. ID. NO.:25), |
| GC box 4: | CTG CCC TTC CCT TCC CTG CCC C | (SEQ. ID. NO.:26), |
| GC boxes 1 + 2: | CCC CAA CCC TGC CCT TAC CTG CCC CTT ACA GCC GGC CAC CTC | (SEQ. ID. NO.:27), |
| CC boxes 3 + 4: | CTT CCC TGC CCT TCC C TT ACC TGC CCC TTA CGG CCC CGC CCG CCC | (SEQ. ID. NO.:28). |

The potential CDE element in the cylin A1 promoter was mutated using the following oligonucleotide: CCA CCT CTT AAC AAG CTT CCT CCA GTG CA (SEQ. ID. NO.:29).

The cyclin Al-EGFP construct was finally constructed by cloning a BgIII-HindIII fragment from the PGL3-Basic-Cyclin A1 Promoter construct into the promoterless EGFP-1 (Clontech) plasmid.

### Example 13

Genomic Cloning and Gene Structure of the Human Cyclin A1 Gene.

A human genomic lambda phage library was screened using the cDNA of cyclin A1 as a probe. Several clones containing pieces of the gene were obtained and one clone with a 14.5 kb insert contained the entire gene. A 2.2 kb fragment at the 5′ end of the gene was subcloned and sequenced. The 2.2 kb fragment contained the first intron and parts of exon 2. The other exon-intron boundaries were analyzed by PCR-amplification and sequencing using sets of primers that span the entire coding region. The human cyclin A1 gene consists of 9 exons and 8 introns which extend over 13 kb.

### Example 14

Analysis of Transcription Start Sites.

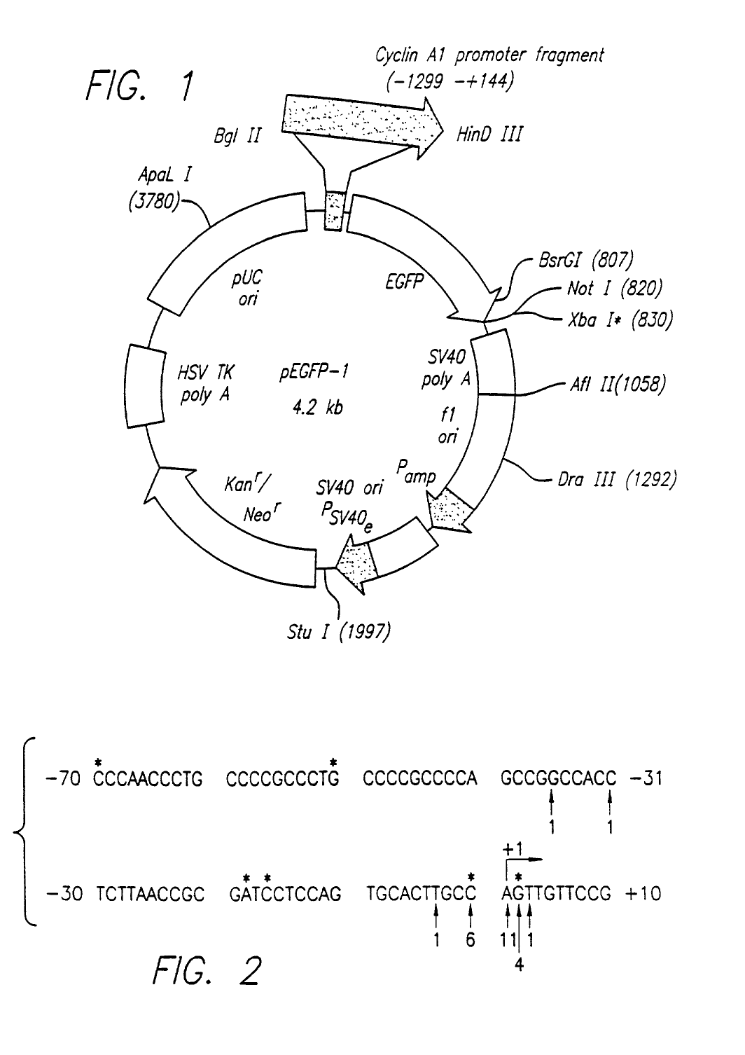

Transcription start sites were determined using primer extension analysis and 5′ RACE. Primer extension was carried out as outlined in Example 12. A sample without RNA and a sample of t-RNA (10 μg) were used as negative controls. The primer extension products shown in FIG. 2 are indicated by an asterisk above the appropriate nucleotide of the indicated sequence. Starting points of the RACE products are indicated by an arrow underneath the sequence. The number of RACE clones (total 25) starting at a particular base is indicated by the number shown below the arrows. The site where 44% (11/25) RACE clones started was assigned +1.

Both methods demonstrated the existence of several transcription start sites. The PCR product from the RACE reaction consisted of a single band of -450 bp. Sequencing of the inserts after cloning revealed that 80% of the RACE products (20/25) started from a 4 base pair stretch, and thus the predominant start site was assigned +1. This site is 130 bp upstream of the translation initiating ATG codon. Primer extension analysis identified the same start sites, but minor products were also seen further upstream (FIG. 2). The major start site coincides with the RACE results of the 5′ end of the cDNA clone described by Yang et al. (1997). Neither RACE clones nor primer extension assays showed evidence for a second transcript in myeloid leukemia cells that could indicate a transcriptional start site upstream of the second ATG in intron 1 (data not shown).

### Example 15

Potential Transcription Factor Binding Sites in the 5′ Upstream Region.

Genomic sequences 1299 bp upstream of the transcription start site were cloned and sequenced. No TATA box was found in proximity to the putative transcriptional start site. The main transcriptional start site is likely to function as an initiator region (Inr) since the sequence “CCAGTT” is very similar to the consensus Inr sequence “TCA G/T T T/C” (T. W. Burke and J. T. Kadonaga, Genes & Development 1:3020-31 [1997]). No DPE element was found downstream of the main transcriptional start site. (See id.). Several potential binding sites for transcription factors occur within the sequence.

FIG. 3 represents the 5′ upstream region of the human cyclin A1 gene. The first bases of the different fragments are indicated, as well as potential transcription factor binding sites between −190 to +144. The transcriptional start site is marked with an arrow and the translational initiation codon is boldfaced. An E2F site is located at nt. +139 to +144 and another possible site starting at +67. A site that resembles the cycle dependent element (CDE) of the cyclin A2 promoter was found at −28. (J. Zwicker et aL, EMBO J. 14:4514-22 [1995]). However, this element was located on the antisense strand. No cell cycle genes homology region (CHR) was found. Potential Myb sites were predicted starting at positions +2, −27 and −66. However, c-myb protein bound only at the first two of these sites. (See FIG. 3 and Example 23). The nucleotide sequences contain two CpG islands of up to 90% GC content reaching from −1000 to −700 and from −550 to −50. Multiple GC boxes are found in this region, and six GC boxes grouped as three double sites are located between nt −150 and −45.

### Example 16

Functional Analysis of the Basal Activity of the Cyclin A1 Promoter.

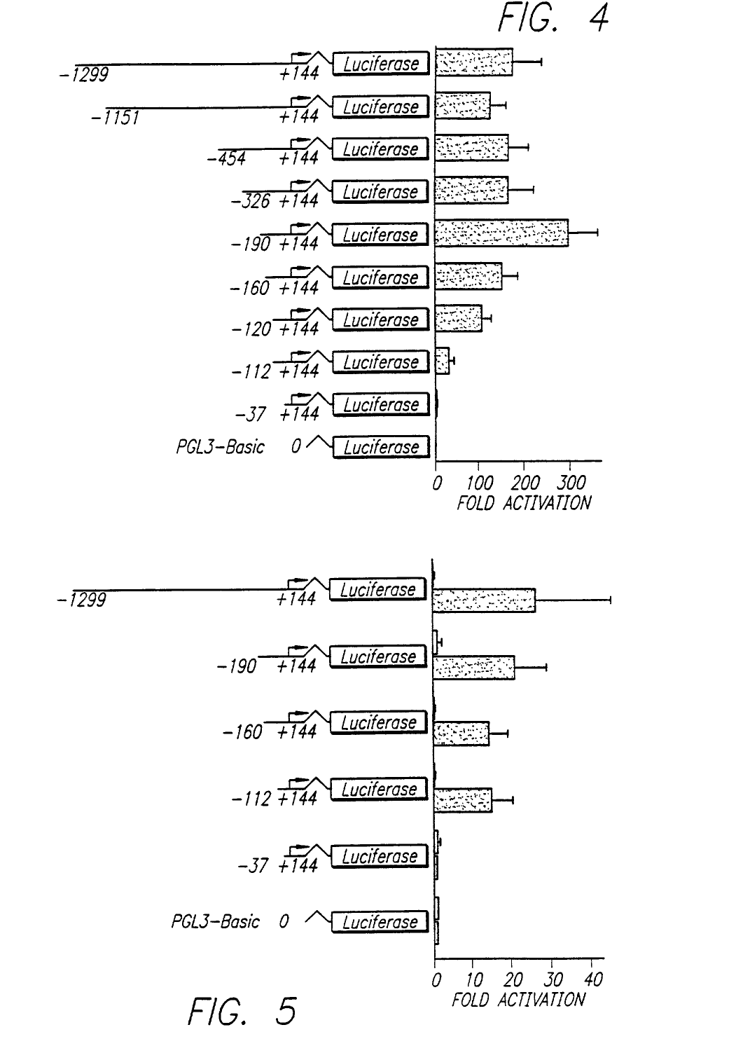

Portions of the cyclin A1 promoter were Pfu-PCR amplified and cloned into the promoterless PGL3-Basic Luciferase vector. Promoter activity was analyzed after transient transfection into Hela cells. FIG. 4 represents transactivation activity of cyclin A1 promoter fragments in Hela cells. Activity of 5′ deletion constructs was analyzed in luciferase assays. Values are expressed as fold activation (PGL3-Basic=1); means and SEM of three independent experiments are shown. The construct containing nucleotides from −1299 to +144 from the 5′ cyclin A1 upstream region showed significant promoter activity when cloned in the sense direction. The same fragment cloned in the opposite direction or a construct containing solely exon 1 and intron 1 did not show promoter activity (data not shown).

Deletions from the 5′ end were made for the −1299 to +144 fragment using exonuclease III treatment. Transient transfection and subsequent luciferase assays revealed the strongest activity occurred in the construct containing the fragment from −190 to +144 bp. (FIG. 4). Both the −1299 to +144 and the −190 to +144 constructs exhibited promoter activity in a variety of cell lines including Cos-7(monkey kidney cell), MCF-7 (breast cancer cell), U937 (myeloid leukemia cell), KCL22 (myeloid leukemia cell), PC3 (prostatic cancer cell), Hela (cervical cancer cell) and Jurkat (T-cell lymphoma). (Data not shown). In all of these mammalian cell lines, luciferase activities generated by the -190 to +144 construct were higher than those by the -1299 to +144 construct. Constructs with a 5′ end containing less than 190 bp upstream of the transcription start site showed a progressive loss of promoter activity. A construct containing bp -37 to +144 showed only two-fold higher activity than the promoterless vector PGL3-Basic.

### Example 17

Role of Sp1 and GC Boxes for Transcriptional Activity of the Cyclin A1 promoter.

TATA-less promoters frequently depend on GC boxes to activate transcription. (J. Lu et al., J. Biol. Chem. 269:5391-5402 [1994]; M. C. Blake et al., Molec. Cell. Biol. 10:6632-41 [1990]). One of the main classes of transcription factors binding to these sites are Sp1 family proteins (A. J. Courey and R. Tjian, Cell 55:887-98 [1988]; A. P. Kumar and A. P. Butler, Nucleic Acids Res. 25:2012-19 [1997]; G. Hagen et al., J. Biol. Chem. 270:24989-94 [1995]). The cyclin A1 promoter contains at least six potential GC boxes between 190 and 37 bp upstream of the transcription start site. The importance of Sp1 for the activity of the cyclin A1 promoter, was demonstrated by the use of various promoter constructs that were transfected into the Drosophila cell line S2, which lacks endogenous Sp1 and Sp3.

FIG. 5 shows activity of the cyclin A1 promoter fragments in the Drosophila cell line S2. Activity is indicated as fold activation of PGL3-Basic as compared to reporter gene activity without addition of Sp1 expression plasmid. The punctated and solid bars represent activities without and with Sp1 co-expression, respectively. When transfected alone, the activity of all cyclin A1 promoter fragments was not significantly different from the empty vector control. (FIG. 5, dotted bars).

The addition of a Sp1 expression plasmid strongly activated transcription by 15- to 25-fold from the cyclin A1 promoter. (FIG. 5, solid bars). Increased transcriptional activity was observed only for constructs containing sequences starting between −1299 and -112 bp upstream of the transcription start site. The construct containing the nucleotide sequences between −37 and +144 did not show any increase in activity, implying that SpI binding sites between −112 and −37 are essential for Sp1 mediated transcriptional activity of the cyclin A1 promoter in Drosophila cells. This region contains four GC boxes which are grouped in two pairs. (FIG. 3).

The ability of Sp1 and other Sp1 family members to bind to these sites was shown by gel-shift experiments performed using 5 μg Hela nuclear extract. Complexes bound to a ³²P-end labeled oligonucleotide containing GC box Nos. 1 and 2, and the labeled oligonucleotide containing GC boxes Nos. 3+4. Binding was competed away with a 100-fold excess of cold Sp1 consensus oligonucleotide and by a 100-fold excess of cold oligonucleotides using either GC box Nos. 1+2 or GC box Nos. 3+4. A 100-fold excess of a non-specific oligonucleotide did not alter specific complex binding. Antibodies against Sp 1 were added to some samples, and antibodies against Sp3 were present in reactions in others. These supershift experiments with antibody against either Sp1 or Sp3 demonstrated the presence of Sp1 in one complex, and the presence of Sp3 in two other complexes. (Data not shown).

The relevance of the GC boxes for promoter activity was further studied by mutational analysis. Point mutations were made in each GC box. Each mutant was tested either alone with the remaining sites unaltered or in combination with the other mutant sites. Luciferase analyses demonstrated that a mutation in either GC box No. 1 or 2 reduced promoter activity by about 40 and 75%, respectively, whereas a single mutation of either GC box No. 3 or 4 did not have a major effect on promoter activity.

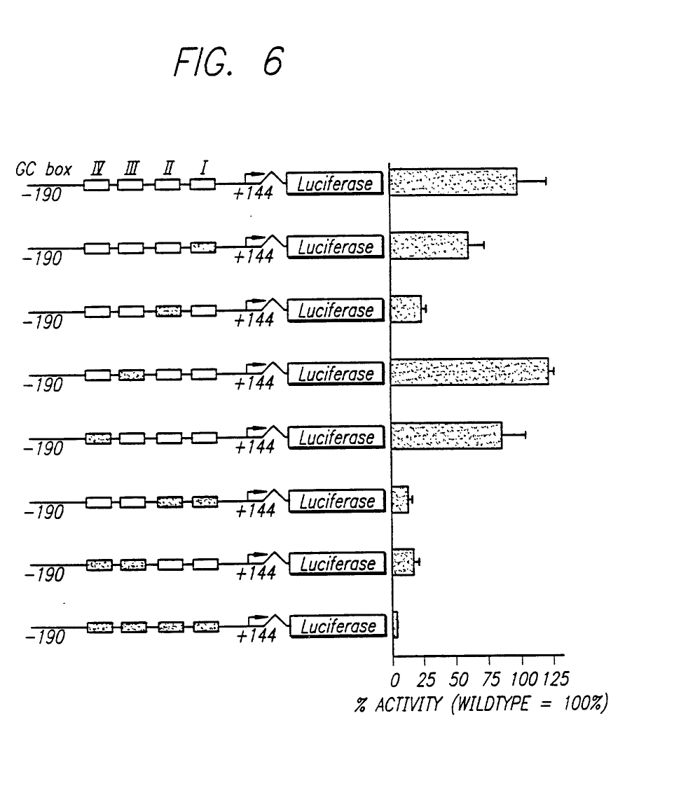

FIG. 6 shows effects of GC box mutations on promoter activity. Individual GC boxes or their combinations were mutated and transiently transfected into Hela cells. Activity of the wild type construct containing nt −190 to +144 was set as 100%. Wild type GC boxes are indicated in white and mutated GC boxes are shown in black. Mutation of GC Box Nos. 1 and 2 together, decreased promoter activity by 85%. The presence of at least one of the two upstream GC boxes (GC Box Nos. 3 or 4) being intact was essential for cyclin A1 promoter activity, as mutations in both reduced promoter activity by about 80%. Mutations of all four GC boxes reduced activity of the cyclin A1 promoter by 95%.

### Example 18

Cell Cycle Regulation of Promoter Activity.

The concentration of cyclins vary during the cell cycle, and one mechanism of their regulation occurs at the transcriptional level. (R. Muller, Trends in Genetics 11:173-78 [1995]). To analyze cell cycle regulation of promoter activity, transiently transfected cells were arrested in different phases of the cell cycle and subsequently analyzed for luciferase activity. Cell cycle regulated activity was found for the full length promoter as well as for the construct containing the −190 to +144 fragment.

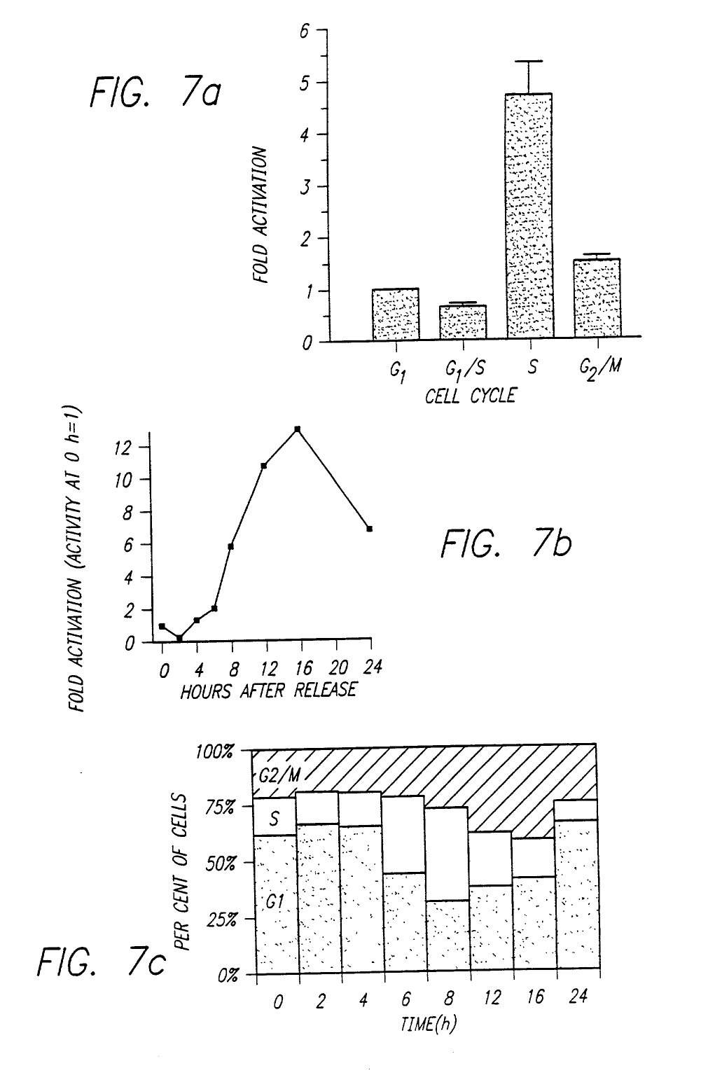

FIG. 7 shows Cell cycle regulated activity of the cyclin A1 promoter in Hela cells. In FIG. 7(A), Hela cells were cell cycle arrested after transfection with a luciferase construct containing nt −190 to +144 of the cyclin A1 promoter. Cells were subsequently analyzed for luciferase activity. Cell cycle synchronization was confirmed by flow cytometry (data not shown). The bars represent means and SEM of at least three independent experiments. Promoter activity at 0 h was set as 1. The cyclin A1 promoter activity was relatively low during the G₀/G₁ phase. It increased after the cell cycle progressed beyond the G₁/S boundary.

In FIG. 7(B), Hela cells were synchronized at the G₁/S boundary using aphidicolin, following transient transfection and serum starvation. Cells were released from the block and harvested at the indicated time points for luciferase and cell cycle analyses. The graph depicts data from a representative experiment. When transiently transfected Hela cells were released from an aphidicolin block, luciferase values started to increase after 6 hours and reached a maximum after 12-16 h.

FIG. 7(C) shows cell cycle distribution at the different time points of the time-release experiment. The hatched, open and solid bars represent G₁, S and G₂/M phases, respectively. The highest levels of activity were observed in the S and G₂/M phases. The maximum promoter activity corresponded to the percentage of cells present in the S and G₂/M phases. This is consistent with data showing that levels of cyclin A1 mRNA accumulate during S phase, with the highest levels present at the S and G₂/M phases. (Yang et al., Mol. Cell. Biol. [in press 1999]).

Fragments containing nucleotides −1299 to +144, −190 to +144, or −190 to +13 performed similarly in all these experiments (data not shown).

Various point mutations and deletions were generated in the presumed E2F sites and the suspected CDE element in order to define the regions that are relevant for cell cycle regulation of the cyclin A1 promoter. Activity of the wild type construct (containing the -1299 to +144 fragment) in aphidicolin arrested cells was set as 1.0 and compared to the other constructs. Only a 40% decrease was detected for the construct containing the four mutated GC boxes. Nucleotides in the suspected CDE in antisense direction were mutated in the construct called mutation −19 to −24. There was a strong increase in promoter activity after release from a G₁/S block by aphidicolin. Constructs lacking the four GC boxes either due to mutation or 5′ deletion were not induced upon entering the S phase. No significant difference was observed between wild type and the mutation −19 to −24 construct.

These findings are consistent with repression of Sp1 mediated activity in the G1 phase of the cell cycle. Selective repression of Sp1 mediated activity by Sp3 has been demonstrated to be relevant in cell cycle regulated promoters containing several Sp1 sites. The dihydrofolate reductase (DHFR) promoter contains four Sp1 sites and is specifically repressed by Sp3. (M. J. Birnbaum et al., Biochemistry 34:16503-08 [1995]). Besides repression by Sp3, other mechanisms probably contribute to repression of the cyclin A1 promoter in G1. Studies have shown that repression of glutamine rich activators such as Sp1 and NF-Y is the predominant mechanism of cell cycle regulation for several promoters (J. Zwicker et al., (1995) Nucleic Acids Research 23:3822-30 [1995]; J. Zwicker et al., (1998) Nucleic Acids Research 26:4926-4932 [1998]). However, none of the known repressor elements (CDE, CHR, E2F) appears to be relevant for the cyclin A1 promoter.

A 3′ deletion construct (−190 to +13) was generated by PCR that deleted the two presumed E2F sites downstream of the transcriptional start site. Mutations in these two presumed E2F sites, the mutation in the inverted presumed CDE element, and the 3′ deletion showed an indistinguishable pattern of cell cycle regulation when compared to the wildtype. (Data not shown).

Hence, these E2F sites and the inverted CDE element are unlikely to play a role in cell cycle regulation of the promoter. Analysis of 5′ deletions and the constructs containing the mutated GC boxes revealed that the four GC boxes are essential for cell cycle regulation. The activity of the construct containing the mutated GC boxes showed 60% of the activity of the wild type reporter construct in G₁ phase. However, the activity of the construct failed to increase when cells entered S phase and showed only 4% of the wild type cyclin A1 promoter activity. Similar data were obtained for the 5′ deletion lacking the four GC boxes.

### Example 19

Screening Transgenic Vertebrates for the Presence of Cyclin A1-EGFP DNA

Transgenic mice were screened by PCR-amplification of DNA sampled from their tails. The mice were anesthetized with metafane, and a 1-cm piece of tail tip was cut using a sterile scalpel. The tail biopsy was incubated with 100,ug of Proteinase K in 700 μL lysis buffer (10 mM Tris, pH7.5, 1 mM EDTA, and 10% SDS) overnight at 50° C. The lysate was extracted once with 500 μl phenol, twice with phenol/chloroform (1:1) and was precipitated with ice cold isopropanol. The precipitate was centrifuged and the pellet was washed once with 70% ethanol. The pellet was allowed to air dry for 30 minutes at room temperature and was then resuspended in 200 μL 10 mM Tris, pH 7.5, 0.1 mM EDTA. The tail DNA was allowed to incubate at 65° C. for 10 min, and it was then stored at 4° C.

For each sample, 100 ng of tail DNA was added to a PCR cocktail mix in a total volume of 50 μL. For each sample tube, the PCR cocktail contained 10 μL of Qiagen Q buffer, 5 μL of PCR buffer (Qiagen), dNTPs and a pair of EGFP-specific primers, 5′-TTG TCG GGC AGC AGC ACG GGG CCG-3 ′ (SEQ. ID. NO.:30) and 5′-TCA CCG GGG TGG TGC CAT CCT TGG-3′ (SEQ. ID. NO.:3 1). A 600 bp fragment was amplified. A positive control contained the cyclin A1-EGFP plasmid DNA, and a negative control contained no DNA.

### Example 20

Selectable Fluorescent Vertebrate Germ Cells Expressing EGFP by the Cyclin A1 Promoter

Five lines of transgenic mice were generated that contain DNA construct pCyclinA1-EGFP-1 and express the flourescent green reporter gene (EGFP) under the control of the cylin A1 promoter (cyclin A1-EGFP mice). Flourescent green protein is seen in male germ cells with FITC filter. The mice were transfected with a construct containing a 1.4 kb 5′ flanking region DNA of human cyclin A1 including, nt. −1299 to +144, inserted into the BgIII/HindIII site of the promoterless fluorescent green protein (EGFP) expression vector pEGFP-1 (Clontech; FIG. 1). The vector also contained a SV40 splice and polyadenylation signal 3′ to the EGFP gene, as well as kanamycin and neomycin resistance genes for selection purposes. The pCyclinA1-EGFP-1 construct was expressed in Cos-7, MCF-7, and U937 cells in vitro. For the generation of transgenic mice, the vector sequences were removed from the construct, and the DNA fragment which comprised the cyclin A1 promoter, the EGFP gene, and the SV40 splice and polyadenylation signal was purified on a 10%-40% sucrose gradient. One-milliliter fractions were collected from the gradient, and the fraction containing the construct was dialized in a slide cassette dialysis membrane (Pierce) against 4 liters of 10 mM Tris, pH 7.5, 0.1 mM EDTA for 48 hours with 3 changes.

The purified pCyclinA1-EGFP construct was used to generate transgenic mice by nicroinjection of DNA into the pronucleus of fertilized eggs. (Gordon and Ruddle, 1980; Hogan, Costantini and Lacy, 1996). The surrogate mothers delivered 38 pups, 8 (21%) of which had integrated the transgene as was shown by PCR and Southern Blot analysis. Two of the founder animals failed to breed and one did not show expression of the transgene in the testis. The remaining 5 animals expressed EGFP in male germ cells.

### Example 21

FACS Analysis of Testicular Cells from Transgenic Mice

Testicular cell suspensions from cyclin A1-EGFP transgenic mice were made using an enzymatic digestion method modified from Dym et al. (M. Dym et al., Biol. Reprod. 52:8-9 [1995]). Testes were dissected from euthanized transgenic mice and decapsulated. The seminiferous tubules were spread apart in Enzyme Mix I: Collagenase I (1 mg/mL; bovine pancreatic, Sigma) in modified HTF medium (Irvine Scientific) containing 1% BSA (1 mg/mL; Sigma, embryo tested) and incubated for 10 min at 37° C. This first enzymatic step is aimed at eliminating cells external to the seminiferous tubules, such as Leydig cells.

The tubules were then lifted into 1 mL of Enzyme Mix II: Collagenase I (1 mg/mL; bovine pancreatic), trypsin type III (50 mg/mL; Sigma), DNAase I (1 mg/mL; Sigma) in modified HTF medium, which contained BSA (1 mg/mL), and the tubules were cut into small pieces using sterile needles attached to 1 mL syringes. The cut tubules were incubated in Enzyme Mix II for 15 min at 37° C. After this incubation, the cells were washed 3 times in 10 mL of the modified HTF by centrifugation at 2,000 rpm and were resuspended. The cells were resuspended in 2 mL of modified HTF and were filtered through 70 μm mesh (Corning) to be analyzed by FACS. Typically, about 3×10⁷ testicular cells were harvested from a mature male mouse. The cells were tested for viability with trypan blue. Kidney cells were prepared in the same way except that the first collagenase incubation was omitted.

The transgenic testicular cells were analyzed for fluorescence and for sideward scatter using a Becton-Dickenson cell sorter on channel 1 (FITC for Green Fluorescent Protein). Based on these properties, four populations were distinguishable: 1) a EGFP-negative population; and populations 2 through 4, which had increasing fluorescence and scatter properties reflecting different cell types.

The cyclin A1-EGFP cells were also tested with PE conjugated PE anti-c-kit antibodies and analyzed with FACS. The FACS analysis showed that there is a population of fluorescent cells which expresses EGFP under the cyclin A1 promoter and that these cells are positive for c-kit. Some of the c-kit cells were not EGFP positive.

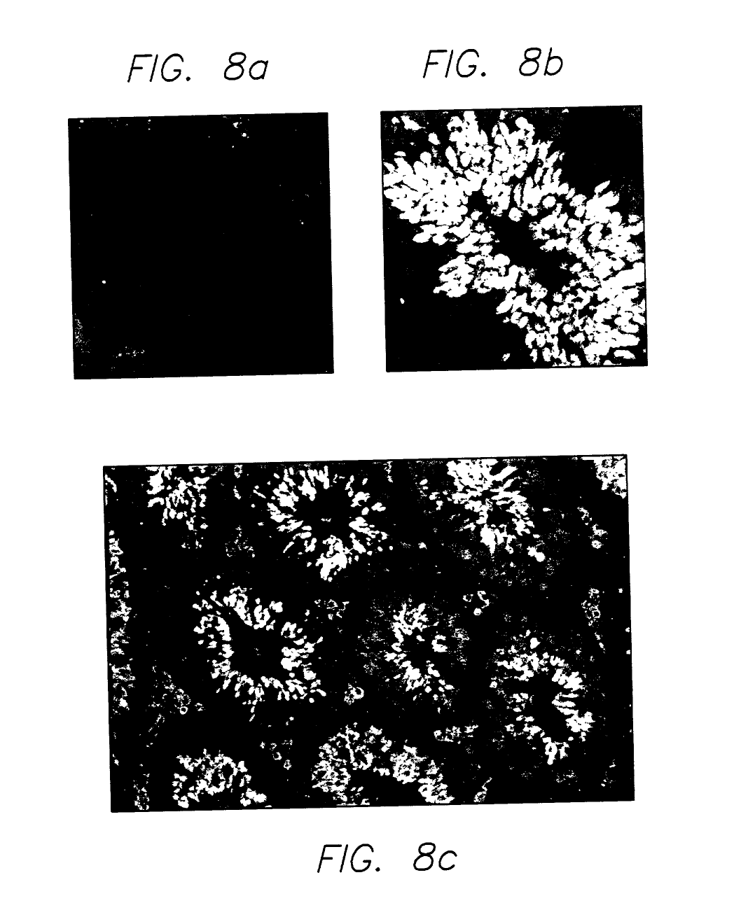

FIG. 8 shows frozen sections from testis of adult mice that were cut, rinsed in phosphate buffered saline (PBS) for 10 min and analyzed by confocal laser scanning microscopy. Whereas no fluorescence could be observed in testicular tubuli of control mice (FIG. 8a), strong and highly specific expression of EGFP (FIG. 8b and c) was detected in testis of transgenic mice. Maximal EGFP expression was observed during and after the first meiotic division and a weaker staining was present in spermatogonia. Magnifications are 400× (FIG. 8a and b) and 100× (FIG. 8c).

### Example 22

The effect of CpG methylation of the cyclin A1 promoter.

Bisulfite sequencing was carried out according to the method described by Clark et al. with minor modifications. (S.J. Clark et al., High sensitivity mapping of methylated cytosines. Nucleic Acids Res 22: 2990-2997 [1994]). Ten mg of DNA was incubated with the bisulfite/hydroquinone solution for six hours. A nested PCR was performed (detailed Primer information will be provided on request) and the final PCR product (ca. 400 bp) was gel purified. The PCR products were either blunt end cloned and at least 10 clones were sequenced, or the purified PCR product was directly sequenced using ³³P-cycle sequencing of nucleotides.

In Vitro Methylation and Luciferase Assay.

The cyclin A1 promoter—luciferase reporter construct was in vitro methylated by SssI following the recommendations of the manufacturer (New England Biolabs). (S. Kudo, Methyl-CpG binding protein MeCP2 represses Sp1-activated transcription of the human leukosialin gene when the promoter is methylated, Mol. Cell. Biol. 18:5492-99 [1998]). S2 Drosophila cells were transfected as described previously using 1 μg of methylated or mock-methylated luciferase—reporter plasmid, 100 ng of Sp1 expression plasmid and 1 μg of a CMV-β-galactosidase expression plasmid used for standardization purposes. One μg of human MeCP2 expression vector or empty vector control were co-transfected. (S. Kudo [1998]). Luciferase experiments were performed in duplicate and independently repeated three times. The human MeCP2 expression vector was a kind gift from Dr. S. Kudo, Hokkaido Institute, Sapporo, Japan.

As described above, the cyclin A1 promoter is highly GC rich and bears a CpG island that extends over several hundred base pairs and ends 50 base pairs upstream of the main transcriptional start site. When the methylation pattern of the CpG dinucleotides in the critical parts of the promoter was analyzed using bisulfite sequencing (S. J. Clark et a. [1994]), a high degree of CpG methylation was observed in somatic, adherent cell lines but not in cyclin A1 expressing leukemia cell lines. Hypomethylation in the leukemic cell lines was clearly restricted to the CpG island since a CpG at nt. +114 outside of the CpG island was found to be completely methylated in all cell lines tested.

To analyze whether methylation of the cyclin A1 promoter was associated with gene silencing, MG63 osteosarcoma cells were stably transfected with a Cyclin A1 promoter—EGFP construct. After prolonged culture of cells (2 months), there were two populations of neomycin-resistant cells, i.e., that showed stable integration of the transgene. One part of the population maintained relatively high expression of EGFP (FIG. 9a, left hand peak), while a fraction of the population ofneomycin-resistant cells lost EGFP expression over time. (FIG. 9a, right hand peak). Both EGFP-expressing and non-expressing cell populations were sorted by flow cytometry and analyzed for CpG methylation of the cyclin A1 promoter transgene. Specific primers were designed for the promoter-EGFP construct to avoid analysis of the endogenous cyclin A1 locus. The transgenic cyclin A1 promoter was substantially non-methylated in the expressing cells (FIG. 9a, left hand sequencing ladder), but cells that had lost EGFP expression showed strong methylation of the cyclin A1 promoter. (FIG. 9a, right hand sequencing ladder).

Studies recently have shown that the methyl CpG binding protein 2 (MeCP2) is an important mediator of methylation-induced gene silencing. (P. L. Jones et al., Methylated DNA and MeCP2 recruit histone deacetylase to repress transcription, Nature Genetics 19: 187-91 [1998]); X. Nan et al., Transcriptional repression by the methyl CpG-binding protein MeCP2 involves a histone deacetylase complex, Nature 393:386-89 [1998]; X. Nan et al., MeCP2 is a transcriptional repressor with abundant binding sites in genomic chromatin, Cell 88:471-78 [1997]). MeCP2 binds specifically to methylated DNA and recruits co-repressors, such as mSin3A, leading to the deacetylation of histones and repression of transcriptional activity. (X. Nan et al. [1997] and [1998]). The human leukosialin gene is one of the genes shown to be negatively regulated by MeCP2 when its promoter is methylated. (S. Kudo [1998]). Leukosialin (similar to cyclin A1) is tissue-specifically expressed in hematopoietic cells and its transcriptional activity depends on the Sp1 transcription factor. Using S2 Drosophila cells that do not express endogenous MeCP2, it was analyzed whether co-transfected MeCP2 would suppress activity of the methylated (+) or unmethylated (−) cyclin A1 promoter (FIG. 9b). Upon transfection of in vitro methylated cyclin A1 promoter constructs into Drosophila cells, we noticed 3-fold repression without MeCP2. When MeCP2 was co-expressed with the methylated cyclin A1 promoter constructs, promoter activity was inhibited by 12-fold, indicating that MeCP2 can suppress transcriptional activation of the methylated cyclin A1 promoter. (FIG. 9b). Since methylation appeared to be involved in regulation of the cyclin A1 gene in the mammalian cell lines, it was investigated whether the site of chromosomal integration would determine the patterns of methylation and expression of the transgenic cyclin A1 promoter. Four lines of transgenic mice carried the cyclin A1 promoter—GFP reporter construct, as described above; this was the same nucleic acid construct used to generate the stable MG63 cell line. All lines of transgenic mice showed highly specific expression in the testis resembling the expression pattern previously determined by in-situ hybridization techniques. (FIG. 8; C. Sweeney et al. [1996]). The EGFP expression pattern in testis was indistinguishable among the different lines. The cyclin A1 promoter was able to direct tissue specific expression in the testis independent of the chromosomal integration site. The methylation status of a transgene is thought to be largely determined by either the chromatin structure at the site of integration, the cis-acting sequences in the transgene, and/or the influence of a locus control region. (J. R. Chaillet et al., Parental-specific methylation of imprinted transgene is established during gametogenesis and progresessivly changes during embryogenesis, Cell 66:77-83 [1991]; K. Matsuo et al., An embryonic demethylation mechanism involving binding of transcriptionfactors to replicating DNA, EMBO J. 17:1446-53 [1998]; M. Brandeis etal., Nature 371:435-38 [1994]). Transgene activity has also been reported to be associated with hypomethylation. (E.g., Pikaart et al. [1998]). Analysis of the methylation status of the human cyclin A1 promoter in the testis of four transgenic mouse lines showed that the promoter and the transgene were not methylated in the testis of two lines. However, the promoter and the transgene were heavily methylated in testis of the two other lines. No difference in the EGFP expression pattern in testis could be found between the murine lines either with or without CpG methylation. To confirm that EGFP was highly expressed despite methylation in these male germ cells, testis cells were disaggregated and sorted by flow cytometry as described above.

Bisulfite sequencing confirmed that methylation of the cyclin A1 promoter in germ cells did not inhibit expression of the transgene in testis. One of the murine lines without methylation in testis showed promoter methylation in the kidney and bone marrow, but not in the liver and did not express the transgene in any organ besides the testis. The silencing of a gene in the absence of methylation has been described for other genes as well. (E.g., P. M. Warnecke and S. J. Clark, DNA methylation profile of the mouse skeletal alpha-actin promoter during development and differentiation, Mol. Cell Biol. 19:164-72 [1999]). One transgenic murine line did not show a significant degree of methylation of the transgenic cyclin A1 promoter anywhere and expressed EGFP in a subset of cells in the kidney (25%), spleen (10%) and bone marrow (16%). Taken together, transcriptional activity of the cyclin A1 promoter transgene outside of the testis was only seen when the promoter was not methylated. This finding might supports a linkage of methylation of the cyclin A1 promoter to transcriptional repression in somatic cells. In contrast, methylation of the cyclin A1 promoter—EGFP transgene did not lead to silencing in murine male germ cells.

### Example 23

Transactivation of Cyclin A1 Promoter by c-myb.

Analysis of the cyclin A1 promoter sequence showed potential binding sites for c-myb within the −190 to +144 fragment. (FIG. 3). To analyze further the differences in expression, four human cell lines were chosen that differed in the degree of cyclin A1 expression. Two were derived from myeloid cells (U937, KCL22) and two others from solid carcinomas (PC3 prostate cancer, Hela cervical carcinoma). Expression of cyclin A1 was analyzed by RT-PCR followed by Southern blotting. The RT-PCR results confirmed that cyclin A1 expression differed between the myeloid and the non-myeloid cell lines. The highest RNA levels were found in U937 and the lowest occurred in Hela cells.

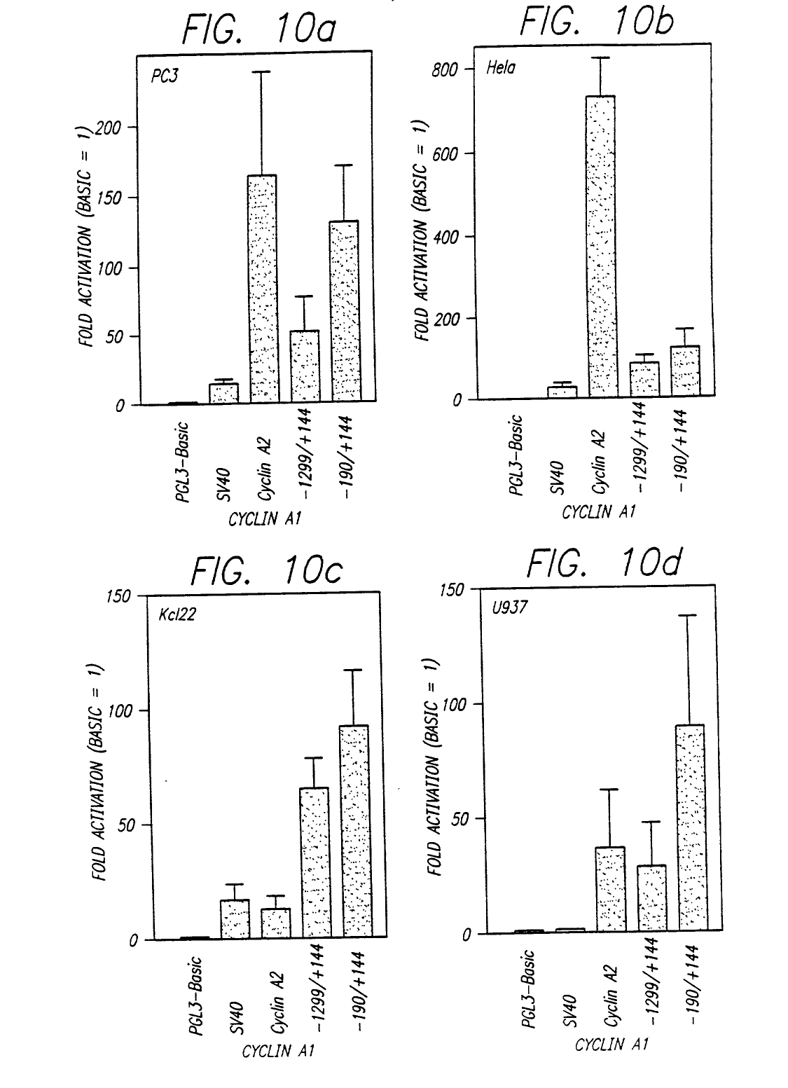

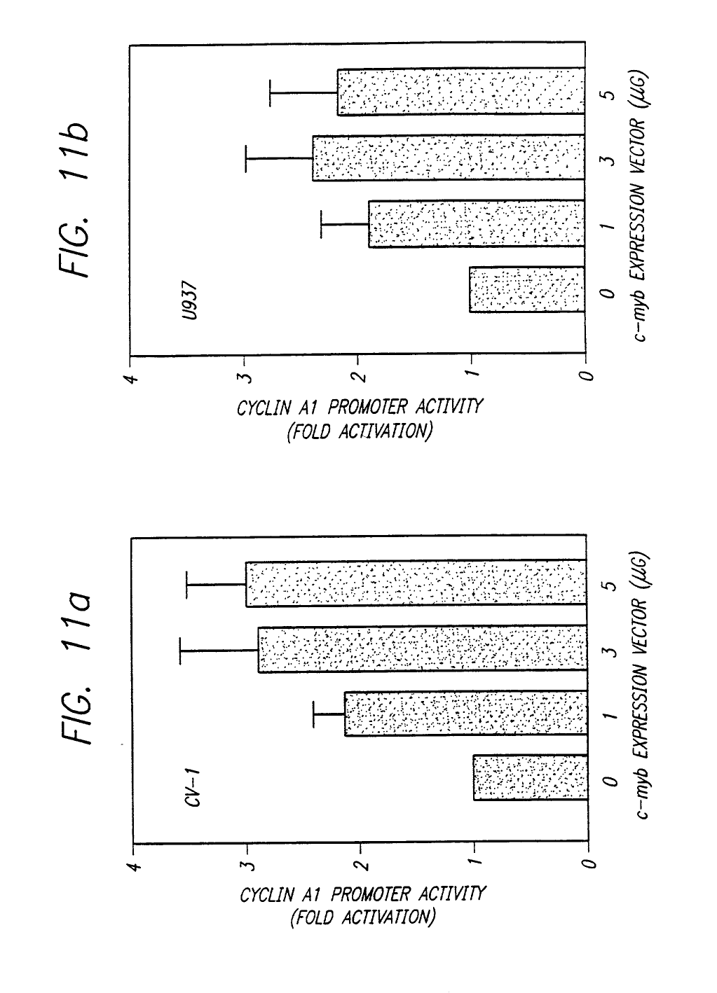

To analyze whether differences in RNA levels could be related to promoter activity, the cyclin A1 promoter was transiently transfected into several myeloid and adherent cells lines (FIG. 10). Both cyclin A1 promoter luciferase constructs ranging from −1299 to +144 and from −190 to +144 showed activity in all four cell lines (FIG. 10). The reporter activity of the shorter promoter fragment was always higher than the activity of the longer fragment. In addition, the activity of the cyclin A1 promoter was higher than that of the SV40 promoter (without enhancer) in all four cell lines.

The cyclin A2 promoter is tightly cell cycle regulated and is assumed to be transactivated in all cycling mammalian cells. Activity of the cyclin A2 promoter was detectable in all four cell lines, but the degree of activity was inversely correlated with the cyclin A1 promoter activity. Cyclin A2 promoter activity was higher in PC3 and Hela cells and it was lower in the myeloid cell lines as compared to the cyclin A1 promoter activity. (FIG. 10). Preferential activity of the cyclin A1 promoter in myeloid cells (compared to the cyclin A2 promoter) was evident for both promoter constructs tested. The inverse relationship between cyclin A2 and cyclin A1 was also present at the RNA level in samples from patients with acute myeloid leukemia. (R. Yang et al. [1999]). However, activity of the cyclin A1 promoter by transient transfection was not limited to the myeloid cell lines but was also present in PC3 and Hela cells. The tissues from which these cell lines derived express very low levels of cyclin A1. An explanation could be that transcription factors expressed in the cell lines, but not expressed in the normal tissue, lead to aberrant promoter activity. One transcription factor expressed in a wide variety of cell lines is c-myb. Western blot analysis demonstrated expression of c-myb in all four cell lines as well as in ML-1, another myeloid cell line that expresses high levels of cyclin A1. The non-myeloid cell lines appeared to have only a high molecular weight form while the myeloid lines had both a high and a low molecular weight form. This may reflect a phosphorylated and a non-phosphorylated myb protein.

To analyze promoter transactivation by c-myb, a c-myb expression vector was transfected (0 to 5 μg of co-transfected plasmid DNA encoding c-myb) along with the -190 to +144 cyclin A1 promoter construct into CV-1 cells that do not express c-myb. A dose-dependent increase in cyclin A1 promoter activity occurred (FIG. 11a), and no increase in activity was observed when c-myb was co-transfected with the empty reporter plasmid (data not shown). The same experiments were repeated using U937 myeloid cells, which express rather low levels of c-myb. As in CV-1 cells, in U937 c-myb clearly transactivated the promoter with maximal activity occurring when 3 μg of c-myb-encoding DNAs were co-transfected. (FIG. 11b). These findings indicate that the cyclin A1 promoter can be transactivated by c-myb in adherent as well as in myeloid cell lines.

To analyze whether c-myb directly affected the cyclin A1 promoter, binding of c-myb protein to the predicted myb binding sites in the promoter region was examined. Gel-shift experiments were performed with c-myb protein expressed in Cos-1 cells and ³²P-labelled oligonucleotides constituting the myb-binding sites of the cyclin A1 promoter. Experiments showed that c-myb was able to bind to the cyclin A1 promoter at the +2 to +5 binding site. Weak binding was seen at the potential myb site at −27 to -24 and no specific binding at the site at position -66 could be detected. Nuclear extracts from Cos-1 cells expressing c-myb led to the appearance of two new bands compared to nuclear extract prepared from Cos-1 cells transfected with empty expression vector, only. Specificity of the binding to the +2 site was confirmed using competitor oligonucleotides and c-myb specific antibody. Addition of c-myb specific antibody led to extinction of both bands. The faster migrating band appeared at the same position as the c-myb band produced on a myb consensus binding site (data not shown). Therefore the slower migrating band might be a complex of proteins with one of them being c-myb. No c-myb binding could be detected using a potential binding site at −66. The binding site at −27 showed a rather weak band after incubation with the c-myb expressing nuclear extract. (Data not shown). Also, the band did not disappear after addition of c-myb antibody implying that c-myb either did not or only weakly bound this site. To test whether c-myb activation of the promoter was affected by alteration of the myb binding sites, different sites were mutated and the resulting constructs were transfected in KCL22 cells. These cells showed the highest c-myb expression of all the cell lines. Abrogation of the myb site at +2 clearly diminished promoter activity by 50% whereas a mutation at either −27 or mutation of the ets site at −15 did not lead to a decrease in promoter activity. The myb site at +2 to +5 is close to the transcriptional start site and the base pairs surrounding the transcriptional start site could function as an Initiator (Inr). To rule out that the observed effects of the mutation at +2 depended on the loss of binding of the basal transcriptional machinery, we transfected the mutated reporter construct together with the c-myb expression plasmid or an empty vector control into CV-1 cells and compared the results with transfections using the wildtype promoter plasmid. The mutation at +2 led to a minor reduction in promoter activity when transfected with the empty vector control. However, transactivation of the mutated reporter plasmid by c-myb was reduced by more than 50%, indicating that c-myb can transactivate the cyclin A1 promoter through this site. Other sites or indirect effects may contribute to the cyclin A1 promoter activation, because the mutation at +2 did not abolish the increase in promoter activity entirely. Different amounts of c-myb were co-expressed with a cyclin A1 promoter construct (−190 to +144 fragment). Empty vector was used to reach the same total amount of DNA in all experiments. Mean and standard error for three independent experiments are shown.

The foregoing examples being illustrative but not an exhaustive description of the embodiments of the present invention, the following claims are presented.

We claim:

1. A method of obtaining a selectable transgenic stem cell of a vertebrate, comprising:
administering to a gonad of a male vertebrate a transfection mixture comprising at least one transfecting agent and at least one polynucleotide comprising a transcriptional unit of a stem cell-specific promoter operatively linked to a DNA encoding a fluorescent or light-emitting protein, under conditions effective to reach a germ cell or germ cell precursor of the male vertebrate; and
causing said polynucleotide to be taken up by, and released into, said germ cell or precursor cell;
incorporating said polynucleotide into the genome of said germ cell or precursor cell, whereby a selectable transgenic stem cell is obtained expressing said fluorescent or light-emitting protein, by which said stem cell can be isolated or selected from a non-stem cell.

2. The method of claim 1, further comprising, after incorporating said polynucleotide into the genome of said germ cell or precursor cell, breeding said male vertebrate with a female of its species to obtain a transgenic progeny expressing said fluorescent or light-emitting protein in at least one of its stem cells.

3. The method of claim 2, wherein breeding is by in vitro or in vivo fertilization of an ovum of said female.

4. The method of claim 1, wherein said stem cell-specific promoter is a human cyclin A1 promoter having a nucleotide sequence (SEQ. ID. NO.:2), or an operative fragment or non-human homologue thereof, or an operative derivative of any of these.

5. The method of claim 1, wherein said polynucleotide further comprises at least one insulator element flanking said transcriptional unit, whereby methylation in vivo of said promoter sequence is substantially prevented.

6. The method of claim 5, wherein at least one of said insulator element(s) is a chicken β-globin insulator element.

7. The method of claim 1, wherein said fluorescent or light-emitting protein is a green fluorescent protein, yellow fluorescent protein, blue fluorescent protein, phycobiliprotein, luciferase, or apoaequorin.

8. The method of claim 1, wherein said vertebrate is a mammal or bird.

9. The method of claim 1, wherein said vertebrate is a human, non-human primate, mouse, rat, rabbit, gerbil, hamster, canine, feline, ovine, bovine, swine, pachyderm, equine, or a farm or marine mammal.

10. The method of claim 1, wherein said vertebrate is a duck, chicken, goose, ostrich, emu, dove, quail, guinea fowl, or turkey.

11. The method of claim 1, wherein said germ cell or precursor cell develops into a maturing male gamete after said polynucleotide is incorporated into the genome of said germ cell or precursor cell.

12. The method of claim 2, wherein a stem cell of said progeny is grown in vitro.

13. The method of claim 12, wherein said stem cell is grown in the presence of an inhibitor of DNA methylation.

14. A selectable transgenic stem cell obtained by the method of claim 1.

15. The selectable transgenic stem cell of claim 14, wherein said stem cell is a pluripotent, multipotent, bipotent, or monopotent stem cell.

16. The selectable transgenic stem cell of claim 14, wherein said stem cell is a spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cell.

17. The selectable transgenic stem cell of claim 14, wherein said stem cell is a selectable transgenic male germ cell.

18. A transgenic non-human vertebrate comprising the stem cell of claim 14.

19. The transgenic non-human vertebrate of claim 18, wherein said vertebrate is a non-human mammal or a bird.

20. Vertebrate semen comprising a maturing male gamete obtained by the method of claim 11.

21. A method of producing a non-human transgenic vertebrate animal line having native germ cells, comprising:
breeding the transgenic non-human vertebrate of claim 18 with a member of the opposite sex of the same species; and selecting progeny for stem cell-specific expression of a xenogeneic fluorescent or light-emitting protein.

22. A method of obtaining a selectable transgenic stem cell of a vertebrate, comprising:
administering to a gonad of a male vertebrate a transfection mixture comprising at least one transfecting agent and at least one polynucleotide comprising a transcriptional unit of a cyclin A1 promoter sequence operatively linked to a DNA encoding a fluorescent or light-emitting protein, under conditions effective to reach a germ cell or germ cell precursor of the male vertebrate; and
causing said polynucleotide to be taken up by, and released into, said germ cell or precursor cell;
incorporating said polynucleotide into the genome of said germ cell or precursor cell, whereby a selectable transgenic stem cell is obtained expressing said fluorescent or light-emitting protein, by which said stem cell can be isolated or selected from a non-stem cell.

23. The method of claim 22, further comprising, after incorporating said polynucleotide into the genome of said germ cell or precursor cell, breeding said male vertebrate with a female of its species to obtain a transgenic progeny expressing said fluorescent or light-emitting protein in at least one of its stem cells.

24. The method of claim 23, wherein breeding is by in vitro or in vivo fertilization of an ovum of said female.

25. The method of claim 22, wherein said cyclin A1 promoter sequence comprises SEQ. ID. NO.:2, or an operative fragment or non-human homologue thereof, or an operative derivative of any of these.

26. The method of claim 22, wherein said polynucleotide further comprises at least one insulator element flanking said transcriptional unit, whereby methylation in vivo of said promoter sequence is substantially prevented.

27. The method of claim 26, wherein at least one of said insulator element(s) is a chicken β-globin insulator element.

28. The method of claim 22, wherein said fluorescent or light-emitting protein is a green fluorescent protein, yellow fluorescent protein, blue fluorescent protein, phycobiliprotein, luciferase or apoaequonn.

29. The method of claim 22, wherein said vertebrate is a mammal or bird.

30. The method of claim 22, wherein said vertebrate is a human, non-human primate, mouse, rat, rabbit, gerbil, hamster, canine, feline, ovine, bovine, swine, pachyderm, equine, or a farm or marine mammal.

31. The method of claim 22, wherein said vertebrate is a duck, chicken, goose, ostrich, emu, dove, quail, guinea fowl, or turkey.

32. The method of claim 22, wherein said germ cell or precursor cell develops into a maturing male gamete after said polynucleotide is incorporated into the genome of said germ cell or precursor cell.

33. The method of claim 23, wherein a stem cell of said progeny is grown in vitro.

34. The method of claim 33, wherein said stem cell is grown in the presence of an inhibitor of DNA methylation.

35. A selectable transgenic stem cell obtained by the method of claim 22.

36. The selectable transgenic stem cell of claim 35, wherein said stem cell is a pluripotent, multipotent, bipotent, or monopotent stem cell.

37. The selectable transgenic stem cell of claim 35, wherein said stem cell is a spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cell.

38. The selectable transgenic stem cell of claim 35, wherein said stem cell is a selectable transgenic male germ cell.

39. A transgenic non-human vertebrate comprising the stem cell of claim 35.

40. The transgenic non-human vertebrate of claim 39, wherein said vertebrate is a non-human mammal or a bird.

41. Vertebrate semen comprising a maturing male gamete obtained by the method of claim 32.

42. A method of producing a non-human transgenic vertebrate animal line having native germ cells, comprising
breeding of the vertebrate of claim 39 with a member of the opposite sex of the same species; and selecting progeny for stem cell-specific expression of a xenogeneic fluorescent or light-emitting protein.

43. A method of obtaining a selectable transgenic stem cell of a vertebrate, comprising:
administering to a gonad of a male vertebrate a transfection mixture comprising at least one transfecting agent and at least one polynucleotide comprising a transcriptional unit of a cyclin A1 promoter sequence operatively linked to a DNA encoding a fluorescent or light-emitting protein, under conditions effective to reach a germ cell or germ cell precursor of the male vertebrate; and
causing said polynucleotide to be taken up by, and released into, said germ cell or precursor cell;
incorporating said polynucleotide into the genome of said germ cell or precursor cell;
allowing said germ cell or precursor cell to develop into a maturing male gamete; and
breeding said male vertebrate with a female of its species to obtain a transgenic progeny expressing said fluorescent or light-emitting protein in at least one of its stem cells, whereby said stem cell can be isolated or selected from a non-stem cell.

44. The method of claim 43, wherein breeding is by in vitro or in vivo fertilization of an ovum of said female.

45. The method of claim 43, wherein said cyclin A1 promoter sequence comprises SEQ. ID. NO. :2, or an operative fragment or non-human homologue thereof, or an operative derivative of any of these.

46. The method of claim 43, wherein said polynucleotide further comprises at least one insulator element flanking said transcriptional unit, whereby methylation in vivo of said promoter sequence is substantially prevented.

47. The method of claim 46, wherein at least one of said insulator element(s) is a chicken β-globin insulator element.

48. The method of claim 43, wherein said fluorescent or light-emitting protein is a green fluorescent protein, yellow fluorescent protein, blue fluorescent protein, phycobiliprotein, luciferase or apoaequorin.

49. The method of claim 43, wherein said vertebrate is a mammal or bird.

50. The method of claim 43, wherein said vertebrate is a human, non-human primate, mouse, rat, rabbit, gerbil, hamster, canine, feline, ovine, bovine, swine, pachyderm, equine, or a farm or marine mammal.

51. The method of claim 43, wherein said vertebrate is a duck, chicken, goose, ostrich, emu, dove, quail, guinea fowl, or turkey.

52. The method of claim 43, wherein a stem cell of said transgenic progeny is grown in vitro.

53. The method of claim 52, wherein said stem cell is grown in the presence of an inhibitor of DNA methylation.

54. A selectable transgenic stem cell obtained by the method of claim 43.

55. The selectable transgenic stem cell of claim 54, wherein said stem cell is a pluripotent, multipotent, bipotent, or monopotent stem cell.

56. The selectable transgenic stem cell of claim 54, wherein said stem cell is a spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cell.

57. The selectable transgenic stem cell of claim 54, wherein said stem cell is a selectable transgenic female or male germ cell.

58. A transgenic non-human vertebrate comprising the stem cell of claim 54.

59. The transgenic non-human vertebrate of claim 58, wherein said vertebrate is a non-human mammal or a bird.

60. A maturing male gamete obtained by the method of claim 43.

61. Vertebrate semen comprising the maturing male gamete of claim 60.

62. A method of producing a non-human transgenic vertebrate animal line having native germ cells, comprising
breeding the vertebrate of claim 58 with a member of the opposite sex of the same species; and selecting progeny for stem cell-specific expression of a xenogeneic fluorescent or light-emitting protein.

63. A method of obtaining a selectable stem cell, comprising:
obtaining a maturing male germ cell from a vertebrate;
transfecting said male germ cell in vitro with at least one polynucleotide comprising a transcriptional unit of a stem cell-specific promoter operatively linked to a DNA encoding a fluorescent or light-emitting protein, in the presence of a gene delivery mixture comprising at least one transfecting agent, at about or below the vertebrate's body temperature and for a transfection-effective period of time;
causing said polynucleotide to be taken up by, and released into said germ cell; and fertilizing an ovum with said germ cell such that a transgenic progeny expressing said fluorescent or light-emitting protein in at least one of its stem cells is obtained, said stem cell(s) being selectable from non-stem cells by detecting light emissions from said stem cell(s).

64. The method of claim 63, wherein fertilizing an ovum is by in vitro or in vivo fertilization.

65. The method of claim 63, wherein said stem cell-specific promoter is a cyclin A1 promoter.

66. The method of claim 63, wherein said cyclin A1 promoter sequence comprises SEQ. ID. NO.:2, or an operative fragment or non-human homologue thereof, or an operative derivative of any of these.

67. The method of claim 63, wherein said polynucleotide further comprises at least one insulator element flanking said transcriptional unit, whereby methylation in vivo of said promoter sequence is substantially prevented.

68. The method of claim 67, wherein at least one of said insulator element(s) is a chicken β-globin insulator element.

69. The method of claim 63, wherein said fluorescent protein is a green fluorescent protein, yellow fluorescent protein, blue fluorescent protein, phycobiliprotein, luciferase, or apoaequonn.

70. The method of claim 63, wherein said vertebrate is a mammal or bird.

71. The method of claim 63, wherein said vertebrate is a human, non-human primate, mouse, rat, rabbit, gerbil, hamster, canine, feline, ovine, bovine, swine, pachyderm, equine, or a farm or marine mammal.

72. The method of claim 63, wherein said vertebrate is a duck, chicken, goose, ostrich, emu, dove, quail, guinea fowl, or turkey.

73. The method of claim 63, wherein a stem cell of said transgenic progeny is grown in vitro.

74. The method of claim 73, wherein said stem cell is grown in the presence of an inhibitor of DNA methylation.

75. A selectable transgenic stem cell obtained by the method of claim 63.

76. The selectable transgenic stem cell of claim 75, wherein said stem cell is a pluripotent, multipotent, bipotent, or monopotent stem cell.

77. The selectable transgenic stem cell of claim 75, wherein said stem cell is a spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cell.

78. The selectable transgenic stem cell of claim 75, wherein said stem cell is a selectable transgenic female or male germ cell.

79. A transgenic non-human vertebrate comprising the selectable transgenic stem cell of claim 75.

80. The transgenic non-human vertebrate of claim 79, wherein said vertebrate is a non-human mammal or a bird.

81. Vertebrate semen comprising the male germ cell of claim 78.

82. A method of producing a non-human transgenic vertebrate animal line having native germ cells, comprising
breeding the vertebrate of claim 79 with a member of the opposite sex of the same species; and selecting progeny for stem cell-specific expression of a xenogeneic fluorescent or light-emitting protein.

83. A method of obtaining a selectable stem cell, comprising:
obtaining a maturing male germ cell from a vertebrate;
transfecting said male germ cell in vitro with at least one polynucleotide comprising a transcriptional unit of a cyclin A1 promoter operatively linked to a DNA encoding a fluorescent or light-emitting protein, in the presence of a gene delivery mixture comprising at least one transfecting agent, at about or below the vertebrate's body temperature and for a transfection-effective period of time; and
allowing said polynucleotide to be taken up by, and released into said germ cell;
fertilizing an ovum with said germ cell such that a transgenic progeny expressing said fluorescent or light-emitting protein in at least one of its stem cells is obtained, said stem cell(s) being selectable from non-stem cells by detecting light emissions from said stem cell(s).

84. The method of claim 83, wherein fertilizing an ovum is by in vitro or in vivo fertilization.

85. The method of claim 83, wherein said cyclin A1 promoter sequence comprises SEQ. ID. NO.:2, or an operative fragment or non-human homologue thereof, or an operative derivative of any of these.

86. The method of claim 83, wherein said polynucleotide further comprises at least one insulator element flanking said transcriptional unit, whereby methylation in vivo of said promoter sequence is substantially prevented.

87. The method of claim 86, wherein at least one of said insulator element(s) is a chicken β-globin insulator element.

88. The method of claim 83, wherein said fluorescent protein is a green fluorescent protein, yellow fluorescent protein, blue fluorescent protein, phycobiliprotein, luciferase, or apoaequorin.

89. The method of claim 83, wherein said vertebrate is a mammal or bird.

90. The method of claim 83, wherein said vertebrate is a human, non-human primate, mouse, rat, rabbit, gerbil, hamster, canine, feline, ovine, bovine, swine, pachyderm, equine, or a farm or marine mammal.

91. The method of claim 83, wherein said vertebrate is a duck, chicken, goose, ostrich, emu, dove, quail, guinea fowl, or turkey.

92. The method of claim 83, wherein a stem cell of said transgenic progeny is grown in vitro.

93. The method of claim 92, wherein said stem cell is grown in the presence of an inhibitor of DNA methylation.

94. A selectable transgenic stem cell obtained by the method of claim 83.

95. The selectable transgenic stem cell of claim 94, wherein said stem cell is a pluripotent, multipotent, bipotent, or monopotent stern cell.

96. The selectable transgenic stem cell of claim 94, wherein said stem cell is a spermatogonial, embryonic, osteogenic, hematopoietic, granulopoietic, sympathoadrenal, mesenchymal, epidermal, neuronal, neural crest, O-2A progenitor, brain, kidney, pancreatic, liver or cardiac stem cell.

97. The selectable transgenic stem cell of claim 94, wherein said stem cell is a selectable transgenic female or male germ cell.

98. A transgenic non-human vertebrate comprising the stem cell of claim 94.

99. The transgenic non-human vertebrate of claim 98, wherein said vertebrate is a non-human mammal or a bird.

100. Vertebrate semen comprising the male germ cell of claim 97.

101. A method of producing a non-human transgenic vertebrate animal line having native germ cells, comprising
breeding of the vertebrate of claim 98 with a member of the opposite sex of the same species; and selecting progeny for stem cell-specific expression of a xenogeneic fluorescent or light-emitting protein.

102. A nucleic acid construct, comprising a cyclin A1 promoter having nucleotide sequence (SEQ. ID. NO.:2), or an operative fragment or non-human homologue thereof, or an operative derivative of any of these.

103. The nucleic acid construct of claim 102, further comprising said cyclin A1 promoter operatively linked to a nucleotide sequence encoding a fluorescent or light-emitting protein, as a transcriptional unit.

104. The nucleic acid construct of claim 103, wherein said polynucleotide further comprises at least one insulator element flanking said transcriptional unit.

105. The nucleic acid construct of claim 104, wherein at least one of said insulator element(s) is a chicken P-globin insulator element.

106. The nucleic acid construct of claim 103, wherein the encoded fluorescent or light-emitting protein is a green fluorescent protein, yellow fluorescent protein, blue fluorescent protein, phycobiliprotein, luciferase, or apoaequorin.

107. A transgenic vertebrate cell containing the nucleic acid construct of claim 102.

108. A transgenic non-human vertebrate comprising the cell of claim 107.

109. The transgenic non-human vertebrate of claim 108, wherein said vertebrate is a non-human mammal or a bird.

110. The transgenic vertebrate cell of claim 107, wherein said cell is a transgenic stem cell.

111. The transgenic stem cell of claim 110, wherein said stem cell is a pluripotent, multipotent, bipotent, or monopotent stem cell.

112. The transgenic stem cell of claim 110, wherein said stem cell is a spermatogonial, hematopoietic, embryonic, osteogenic, granulopoietic, sympathoadrenal, mesenchymal, epidermal, neuronal, neural crest, O-2A progenitor, brain, kidney, pancreatic, liver or cardiac stem cell.

113. The transgenic stem cell of claim 110, grown in vitro.

114. The transgenic stem cell of claim 113, grownin the presence of an inhibitor of DNA methylation.

115. A transgenic non-human vertebrate comprising the transgenic stem cell of claim 110.

116. The transgenic non-human vertebrate of claim 115, wherein said vertebrate is a non-human primate, mouse, rat, rabbit, gerbil, hamster, canine, feline, ovine, bovine, swine, pachyderm, equine, or a farm or marine mammal.

117. The transgenic non-human vertebrate of claim 115, wherein said vertebrate is a duck, chicken, goose, ostrich, emu, dove, quail, guinea fowl, or turkey.

118. A kit for transfecting a male vertebrate's germ cells, comprising:
a transfecting agent and a polynucleotide comprising a transcriptional unit of a human cyclin A1 promoter sequence having SEQ. ID. NO.:2, or an operative fragment or non-human homologue thereof, or an operative derivative of any of these, operatively linked to a DNA having a nucleotide sequence encoding a fluorescent or light-emitting protein, whereby said kit may be used to transfect said germ cells.

119. The kit of claim 118, wherein the transfecting agent is a liposome, viral vector, transferrin-polylysine enhanced viral vector, retroviral vector, lentiviral vector, or uptake enhancing DNA segment, or a mixture of any of these.

120. The kit of claim 118, wherein the transfecting agent comprises a retroviral vector, adenoviral vector, transferrin-polylysine enhanced adenoviral vector, human immunodeficiency virus vector, lentiviral vector, Moloney murine leukemia virus-derived vector, mumps vector, a DNA segment that facilitates polynucleotide uptake by and release into the cytoplasm of germ cells, or comprises an operative fragment of- or mixture of any of these.

121. The kit of claim 118, wherein the transfecting agent comprises an adenovirus vector having endosomal lytic activity, and the polynucleotide is operatively linked to the vector.

122. The kit of claim 118, wherein the transfecting agent comprises a lipid transfecting agent.

123. The kit of claim 118, wherein the transfecting agent further compris es a male-germ-cell-targeting molecule.

124. The kit of claim 123, wherein the male-germ-cell-targeting molecule is specific for targeting spermatogonia and comprises a c-kit ligand.

125. The kit of claim 118, further comprising an immunosuppressing agent.

126. The kit of claim 125, wherein the immunosuppressing agent is cyclosporin or a corticosteroid.

127. The kit of claim 123, wherein the kit contains at least one additional polynucleotide comprising a nucleotide sequence encoding for expression of a desired trait.

128. The kit of claim 127, wherein the male-germ-cell-targeting molecule is specifically targeted to spermatogonia and comprises a c-kit ligand; and the kit contains at least one additional polynucleotide comprising a nucleotide sequence encoding for expression of a desired trait.

129. The kit of claim 123, wherein the male-germ-cell-targeting molecule is specifically targeted to spermatogonia and comprises a c-kit ligand; and
the DNA having a nucleotide sequence encoding a fluorescent protein is operatively linked to a cyclin A1 promoter, c-kit promoter, B-Myb promoter, c-raf-1 promoter, ATM (ataxia-telangiectasia) promoter, RBM (ribosome binding motif) promoter, DAZ (deleted in azoospermia) promoter, XRCC-1 promoter, HSP 90 (heat shock gene) promoter, or FRMI (from fragile X site) promoter.

130. The kit of claim 118, wherein said polynucleotide further comprises at least one insulator element flanking said transcriptional unit.

131. The kit of claim 130, wherein at least one of said insulator element(s) is a chicken β-globin insulator element.

132. The kit of claim 118, wherein said fluorescent or light-emitting protein is a green fluorescent protein, yellow fluorescent protein, blue fluorescent protein, phycobiliprotein, luciferase, or apoaequorin.
